(19) United States
(12) Patent Application Publication
(10) Pub. No.: US 2025/0064906 A1
(43) Pub. Date: Feb. 27, 2025

(54) NEUROTOXIN COMPOSITIONS FOR USE IN TREATING DIGESTIVE DISORDERS

(71) Applicant: AEON Biopharma, Inc., Irvine, CA (US)

(72) Inventors: Gregory F. Brooks, Irvine, CA (US); Eric Carter, Laguna Beach, CA (US); Adelbert L. Stagg, Irvine, CA (US); Chad Oh, Beverly Hills, CA (US); Andrew M. Blumenfeld, Del Mar, CA (US)

(21) Appl. No.: 18/712,565

(22) PCT Filed: Nov. 21, 2022
(86) PCT No.: PCT/US2022/080244

§ 371 Date: May 22, 2024

Related U.S. Application Data
(60) Provisional application No. 63/282,520, filed on Nov. 23, 2021.

(51) Int. Cl. A61K 38/48; A61K 9/00

(52) U.S. Cl. CPC A61K 38/4893; A61K 9/0019; C12Y 304/24069

(57) ABSTRACT

Disclosed herein are compositions and methods for use in treating digestive disorders.

### CROSS-REFERENCE TO RELATED APPLICATION

The present application claims the benefit of U.S. Provisional Application No. 63/282,520, filed Nov. 23, 2021, the entire contents of which is incorporated by reference herein.

### FIELD

The present Specification relates to the use of neurotoxins administered to specific locations in the digestive tract, for example to treat digestive disorders such as dyspepsia and gastroparesis.

### BACKGROUND

The primary function of the stomach is the temporary storage of ingested food and fluids, together with the preparation of chyme (the acidic fluid which passes from the stomach to the small intestine, consisting of gastric juices and partly digested food) through physical mixing and chemical action, and the controlled release of aliquots of chyme into the duodenum. This initial process of digestion of nutrients and fluids involves the contraction and relaxation of overlapping muscle layers of the stomach, as well as the contraction and relaxation of the pyloric sphincter, and is coordinated via the central nervous system.

Gastroparesis is a complex, debilitating gastric motility disorder with its sufferers facing challenging symptom management. A diagnosis of gastroparesis is based on a documented history of gastroparesis symptoms and signs, together with objectively delayed gastric emptying in the absence of mechanical obstruction or other etiologies. An increasing body of evidence suggests that gastroparesis is also associated with hypertonicity of the fundus and proximal corpus of the stomach, resulting in a failure of receptive relaxation. Cholecystokinin (CCK) is a neuropeptide and gut hormone that regulates pancreatic enzyme secretion and gastrointestinal motility, and acts as a satiety signal. It is released simultaneously from intestinal cells and neurons in response to a meal. The release of CCK may contribute to gastroparesis.

In both idiopathic and disease-related gastroparesis (for example, diabetic gastroparesis), the dysfunction of one or more of these physiologic processes leads to an interrelated symptom complex, including nausea in over 90% of patients, vomiting in 70-90% of patients, and upper abdominal pain in up to 85% of patients.

Thus, a hypertonic pyloric sphincter or proximal duodenum may impair the ordered emptying of chyme from the stomach resulting in impaired gastric emptying, and provides a rationale for targeting injections in this area, for example to reduce hypertonicity. When associated with a failure of relaxation and accommodation of the fundus and cardia of the stomach, impaired gastric emptying may further accentuate early satiety, postprandial fullness, nausea, vomiting and upper abdominal discomfort and pain.

Given the limited efficacy of current treatment options and associated serious side effects, significant research continues to identify alternative therapeutic options for the safe and effective treatment of gastroparesis. Further research and novel treatment options are needed to address the substantial unmet medical needs of gastroparesis.

Botulinum toxin injection has been shown to be effective in treating disorders of smooth muscle hypertonicity in the gastrointestinal tract in some studies. Endoscopic intra-sphincteric injection of the pylorus with botulinum toxin improves symptoms, signs and gastric emptying in some patients with gastroparesis. Botulinum toxin may interfere with hypertonic contractions of the pylorus, resulting in significant relaxation for the improvement of gastric stasis. It is already used for the prevention and treatment of gastric stasis as a complication of esophagectomy associated with vagotomies, oropharyngeal dysphagia, diffuse esophageal spasm, achalasia, refractory gastroparesis and anal fissures. However, there are no specific protocols in terms of injection sites and dose, that have been established for the use of botulinum toxin injections for the treatment of gastroparesis.

Patients with idiopathic or diabetic gastroparesis who are refractory to medical therapy have shown improvement in symptoms and signs with botulinum toxin injection. The improvement can be maintained for a duration of approximately 3-8 months. Patients who had a positive response to the first dose usually continue to respond to repeat injections, and retreatment is often required.

However, current treatments suffer from inconsistent effectiveness as well as short duration of effect. To improve this outcome, successfully treating gastroparesis patients may require an increased neurotoxin dose administered into more and varied injection sites as compared to current methods.

### SUMMARY

The interrelated digestive symptoms such as nausea, vomiting and upper abdominal pain can be further aggravated by antral dysmotility or uncoordinated gastric peristalsis, thus providing the rationale for neurotoxin injections into the duodenum, pyloric region, proximal corpus, and/or fundus.

Disclosed methods combine sensory-blocking and physical effects to achieve desired treatment goals. For example, sensory-blocking effects are achieved in embodiments by decreasing or modulating nerve activity, for example, vagal nerve activity or nerves in the wall of the stomach, the enteric nervous system, through the use of specifically targeted neurotoxin administration. Some patients with diabetic gastroparesis have problems with the vagus nerve due to demyelination. Some patients with idiopathic gastroparesis have abnormal function of the nerves in the wall of the stomach. In embodiments, nausea is reduced by down-regulating vagal nerve feedback from the stomach to brainstem centers. In embodiments, neurotoxin administration into the wall of the stomach can reduce abnormal nerve activity in the stomach wall and alleviate symptoms, such as nausea, post-prandial bloating and fullness after a meal.

Physical effects are achieved by decreasing or modulating muscular activity, for example activity of the muscles of and proximal to the stomach, through the use of specifically targeted neurotoxin administration. For example, in embodiments, pyloric activity is reduced by injecting neurotoxin into the pyloric region to facilitate gastric emptying. In embodiments, fundal distension potential is increased by injecting neurotoxin into the fundus to enhance the “accommodation” response, facilitating relaxation and reducing nausea, post-prandial bloating and fullness after a meal in some patients with upper gastrointestinal symptoms. In embodiments, relaxing the antrum through the use of neurotoxin administration in some patients can reduce nausea as well as post-prandial bloating and fullness. In embodiments, neurotoxin administration to the proximal duodenum in some patients can relieve abnormal coordination of motor function in the duodenum and facilitate flow of food out of the stomach.

In embodiments, disclosed methods comprise treatment of, for example, disease-related gastroparesis such as diabetic gastroparesis, idiopathic gastroparesis, post-operative gastroparesis, post-viral gastroparesis, organic dyspepsia, functional dyspepsia, pyloric spasm, combinations thereof, and the like.

Disclosed embodiments comprise methods of reducing the severity of one or more symptoms of a digestive disorder including abdominal pain or discomfort, episodic stomach swelling, stomach pain, early fullness, abdominal bloating, early satiety, post-prandial fullness, post-prandial satiety, nausea, vomiting, acid reflux, and combinations thereof.

Disclosed herein are compositions and methods comprising neurotoxins, for example Clostridial neurotoxins including botulinum toxins, and the use thereof to treat conditions such as gastroparesis, dyspepsia, and the like, via injection, for example, targeted endoscopic injection into at least one area of the proximal stomach, for example at least one location between (and including) the fundus and the duodenum. Further embodiments comprise injection into at least two locations between (and including) the fundus and the duodenum, or at least three locations between (and including) the fundus and the duodenum, or at least four locations between (and including) the fundus and the duodenum.

For example, disclosed methods comprise targeted endoscopic injection, of at least one of the fundus, proximal corpus, pyloric region, and duodenum, to treat and prevent the recurrence of the signs and symptoms of gastroparesis, and improve quantitative measures of gastric emptying in patients with gastroparesis.

Targeted endoscopic injections to the pyloric region can comprise injection of at least one, two, or three of the pyloric antrum, pyloric sphincter, and pyloric canal. Disclosed injections can comprise, for example, intramuscular injections, submucosal injections, and combinations thereof. Disclosed methods can comprise intra-sphincteric injections, for example of the pyloric sphincter.

Disclosed embodiments comprise the use of electromyography (EMG), for example smooth muscle electromyography (SMEMG) to improve accuracy when administering botulinum toxin. Disclosed embodiments comprise the use of EMG “needle” electrodes. EMG measures the electrical potential generated by muscle cells when the cells are electrically activated. A needle electrode can discharge short electrical pulses to a nerve or tissue and the time taken for the muscle to contract can be measured. Needle electrodes can also be used to “passively” evaluate muscle and/or nerve activity.

### BRIEF DESCRIPTION OF THE DRAWINGS

FIG. 1 shows the stomach and relevant structures therein, including the location of the pyloric sphincter and fundus. The proximal corpus is located just “below” the fundus.

FIG. 2 shows the pathophysiology of gastroparesis and impact on gastric function in diabetic gastroparesis.

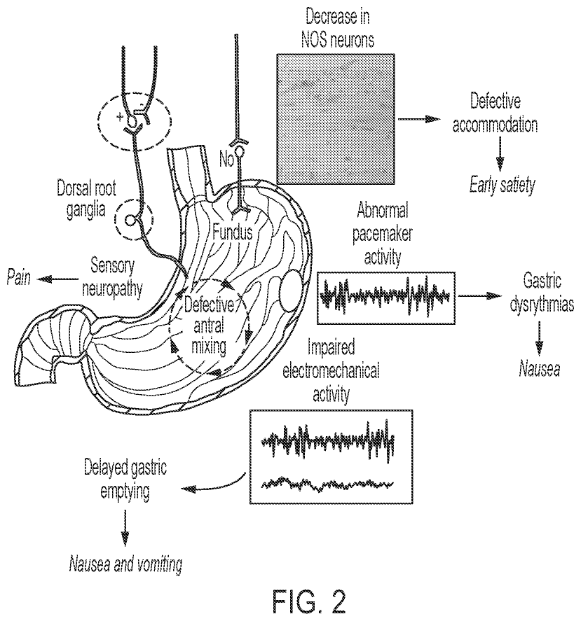

FIG. 3 shows the pathophysiology of gastroparesis and impact on gastric function in diabetic gastroparesis.

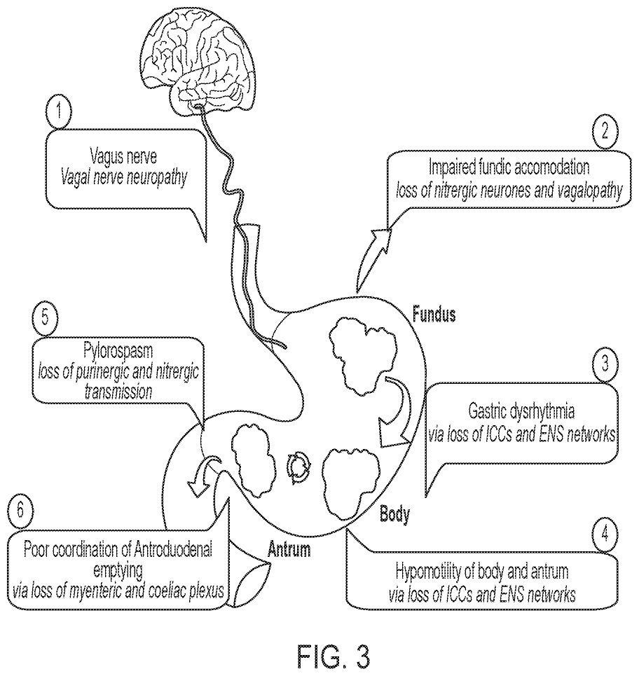

FIG. 4 shows a disclosed 150 Unit injection paradigm (injection sites in blue).

FIG. 5 shows a disclosed 150 Unit injection paradigm (injection sites in blue).

FIG. 6 shows the anatomy of the duodenum.

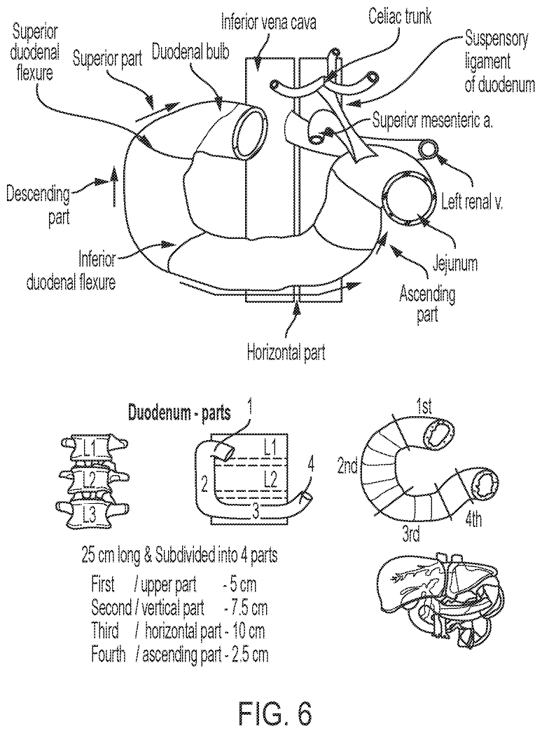

FIG. 7 shows the anatomy of the stomach.

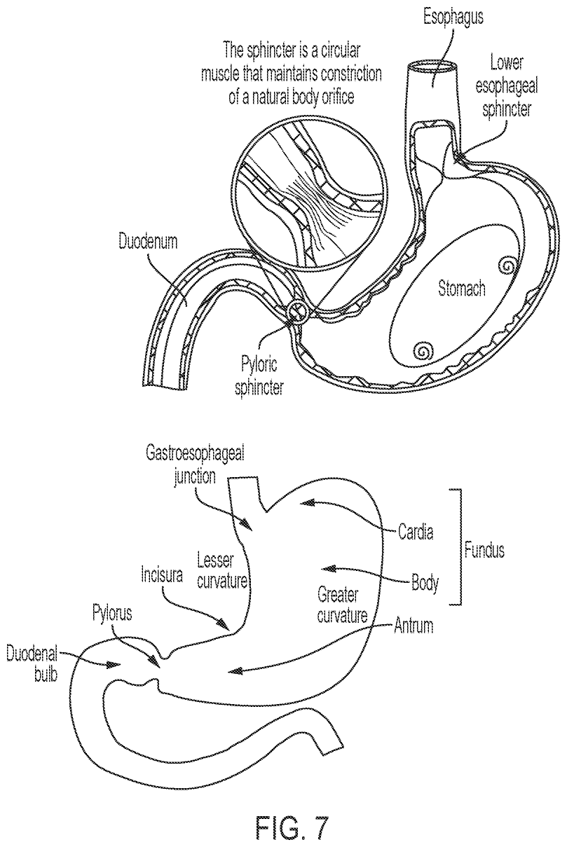

### DETAILED DESCRIPTION

The present disclosure is directed toward methods for reducing the occurrence, recurrence, duration, and/or severity of symptoms associated with digestive disorders such as, for example, disease-related gastroparesis including diabetic gastroparesis, idiopathic gastroparesis, post-operative gastroparesis, post-viral gastroparesis, organic dyspepsia, functional dyspepsia, pyloric spasm, combinations thereof, and the like.

Disclosed methods can comprise neurotoxin, for example botulinum, administration to the pyloric region. For example, disclosed methods can comprise botulinum administration to at least one of the pyloric sphincter, pyloric canal or the pyloric antrum. For example, disclosed embodiments can comprise between 2 and 8 injection sites on the pyloric sphincter. In embodiments, injection to the pyloric sphincter can comprise, for example, administration of a total dose of a neurotoxin, for example via injection of botulinum type A, of 50 to 300 Units. In embodiments, the total volume of the dose is, for example, between 1 and 6 mL.

In embodiments, the number of injections to the pyloric sphincter is between 2 and 6. For example, the injections can be administered at 1 site on each half of the pyloric sphincter, or at 1 site on each quadrant of the pyloric sphincter, or at 3 sites equally spaced on each half of the pyloric sphincter.

Disclosed embodiments can further comprise botulinum administration to the duodenum, for example using a total dose of between 25 and 100 Units. For example, disclosed embodiments can comprise administration via injection to between 2 and 8 sites on the duodenum. For example, 1 injection can be made into the bulb of the duodenum on each side, and between 2 and 6 injections can be made on the duodenum below the bulb no further than 10 cm from the pyloric sphincter.

Disclosed embodiments can further comprise botulinum administration to the fundus, for example using a total dose of 25 and 200 Units. For example, disclosed embodiments can comprise administration via injection at between 2 and 10 sites on the fundus. In embodiments, the injections can be made, for example, in a longitudinal or circumferential pattern spaced between 1 and 4 cm apart.

Disclosed embodiments can further comprise botulinum administration to the proximal corpus.

Disclosed embodiments can further comprise botulinum administration to the fundus and proximal corpus.

Disclosed embodiments can comprise methods comprising EMG such as SMEMG.

Disclosed methods comprise diagnosis of a digestive disorder.

### Definitions

“Administration,” or “to administer” means the step of giving (i.e. administering) a pharmaceutical composition or active ingredient to a subject. The pharmaceutical compositions disclosed herein can be administered via a number of appropriate routs, including intramuscular or subcutaneous routes of administration, such as by injection, topically, or use of an implant.

“Biomarker” means a biological molecule found in blood, other body fluids, or tissue that is a sign of a normal or abnormal process, or of a condition or disease.

“Botulinum toxin” or “botulinum neurotoxin” means a neurotoxin derived from Clostridium botulinum, as well as modified, recombinant, hybrid and chimeric botulinum toxins. A recombinant botulinum toxin can have the light chain and/or the heavy chain thereof made recombinantly by a non-Clostridial species. “Botulinum toxin,” as used herein, encompasses the botulinum toxin serotypes A, B, C, D, E, F, G and H. “Botulinum toxin,” as used herein, also encompasses both a botulinum toxin complex (i.e. the 300, 600 and 900 kDa complexes) as well as pure botulinum toxin (i.e. the about 150 kDa neurotoxic molecule), all of which are useful in the practice of the disclosed embodiments.

“Clostridial neurotoxin” means a neurotoxin produced from, or native to, a Clostridial bacterium, such as Clostridium botulinum, Clostridium butyricum or Clostridium beratti, as well as a Clostridial neurotoxin made recombinantly by a Clostridial or non-Clostridial species.

“Fast-acting neurotoxin” as used herein refers to a botulinum toxin that produces effects in the patient more rapidly than those produced by, for example, a botulinum neurotoxin type A. For example, the effects of a fast-acting botulinum toxin (such as botulinum type E) can be produced within 36 hours.

“Fast-recovery neurotoxin” as used herein refers to a botulinum toxin that whose effects diminish in the patient more rapidly than those produced by, for example, a botulinum neurotoxin type A. For example, the effects of a fast-recovery botulinum toxin (such as botulinum type E) can diminish within, for example, 120 hours, 150 hours, 300 hours, 350 hours, 400 hours, 500 hours, 600 hours, 700 hours, 800 hours, or the like. It is known that botulinum toxin type A can have an efficacy for up to 12 months, and in some circumstances for as long as 27 months, when used to treat glands, such as in the treatment of hyperhidrosis. However, the usual duration of an intramuscular injection of a botulinum neurotoxin type A is typically about 3 to 4 months.

“Neurotoxin” means a biologically active molecule with a specific affinity for a neuronal cell surface receptor. Neurotoxin includes Clostridial toxins both as pure toxin and as complexed with one or more non-toxin, toxin-associated proteins.

“Patient” means a human or non-human subject receiving medical or veterinary care.

“Parenteral administration” and “administered parenterally” are art-recognized terms, and include modes of administration other than enteral and topical administration, such as injections, and include, without limitation, retro-orbital, intraocular, intravenous, intramuscular, intrapleural, intravascular, intrapericardial, intraarterial, intrathecal, intracapsular, intraorbital, intracardiac, intradermal, intraperitoneal, transtracheal, subcutaneous, subcuticular, intra-articular, subcapsular, subarachnoid, intraspinal and intrasternal injection and infusion.

“Pharmaceutically acceptable” or “therapeutically acceptable” refers to a substance which does not interfere with the effectiveness or the biological activity of the active ingredients and which is not toxic to a patient

“Pharmaceutically acceptable carrier” is art-recognized, and includes, for example, pharmaceutically acceptable materials, compositions or vehicles, such as a liquid or solid filler, diluent, excipient, solvent, or encapsulating material, involved in carrying or transporting any subject composition from one organ, or portion of the body, to another organ, or portion of the body. Each carrier must be “acceptable” in the sense of being compatible with the other ingredients of a subject composition and not injurious to the patient. In certain embodiments, a pharmaceutically acceptable carrier is non-pyrogenic. Exemplary materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, sunflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations.

“Pharmaceutical composition” means a formulation in which an active ingredient can be a Clostridial toxin. The word “formulation” means that there is at least one additional ingredient (such as, for example and not limited to, an albumin [such as a human serum albumin or a recombinant human albumin] and/or sodium chloride) in the pharmaceutical composition in addition to a Clostridial (for example, a botulinum neurotoxin) active ingredient. A pharmaceutical composition is therefore a formulation which is suitable for diagnostic, therapeutic or cosmetic administration to a subject, such as a human patient. The pharmaceutical composition can be in a lyophilized or vacuum dried condition, a solution formed after reconstitution of the lyophilized or vacuum dried pharmaceutical composition with saline or water, for example, or as a solution that does not require reconstitution. As stated, a pharmaceutical composition can be liquid, semi-solid, or solid. A pharmaceutical composition can be animal-protein free.

“Purified botulinum toxin” means a pure botulinum toxin or a botulinum toxin complex that is isolated, or substantially isolated, from other proteins and impurities which can accompany the botulinum toxin as it is obtained from a culture or fermentation process. Thus, a purified botulinum toxin can have at least 95%, and more preferably at least 99% of the non-botulinum toxin proteins and impurities removed.

“Pyloric region” means the tissue region between the duodenum and the pyloric antrum, including the pyloric antrum, pyloric sphincter, and pyloric canal.

“Reducing”, “suppressing” and “inhibiting” have their commonly understood meaning of lessening or decreasing.

“Therapeutic formulation” means a formulation that can be used to treat and thereby alleviate a disorder or a disease and/or symptom associated thereof.

“Therapeutically effective amount” means the level, amount or concentration of an agent (e.g. such as a Clostridial toxin or pharmaceutical composition comprising clostridial toxin) needed to treat a disease, disorder or condition without causing significant negative or adverse side effects.

“Treat,” “treating,” or “treatment” means an alleviation or a reduction (which includes some reduction, a significant reduction, a near total reduction, and a total reduction), resolution, or prevention (temporarily or permanently) of a symptom, disease, disorder or condition, so as to achieve a desired therapeutic or cosmetic result, such as by healing of injured or damaged tissue, or by altering, changing, enhancing, improving, ameliorating and/or beautifying an existing or perceived disease, disorder or condition.

“Unit” or “U” means an amount of active botulinum neurotoxin standardized to have equivalent neuromuscular blocking effect as a Unit of commercially available botulinum neurotoxin type A (for example, Onabotulinumtoxin A (BOTOX®)).

### Neurotoxin Compositions

Embodiments disclosed herein comprise neurotoxin compositions. Such neurotoxins can be formulated in any pharmaceutically acceptable formulation in any pharmaceutically acceptable form. The neurotoxin can also be used in any pharmaceutically acceptable form supplied by any manufacturer. Disclosed embodiments comprise use of Clostridial neurotoxins.

The Clostridial neurotoxin can be made by a Clostridial bacterium, such as by a Clostridium botulinum, Clostridium butyricum, or Clostridium beratti bacterium. Additionally, the neurotoxin can be a modified neurotoxin; that is a neurotoxin that has at least one of its amino acids deleted, modified or replaced, as compared to the native or wild type neurotoxin. Furthermore, the neurotoxin can be a recombinantly produced neurotoxin or a derivative or fragment thereof.

In disclosed embodiments, the neurotoxin is formulated in unit dosage form; for example, it can be provided as a sterile solution in a vial or as a vial or sachet containing a lyophilized powder for reconstituting in a suitable vehicle, such as saline for injection.

In embodiments the neurotoxin, for example botulinum toxin, is formulated in a solution containing saline and pasteurized Human Serum Albumin (HSA), which stabilizes the toxin and minimizes loss through non-specific adsorption. The solution can be sterile filtered (0.2 μm filter), filled into individual vials, and then vacuum-dried to give a sterile lyophilized powder. In use, the powder can be reconstituted by the addition, for example, of sterile unpreserved normal saline (sodium chloride 0.9% for injection).

In an embodiment, botulinum type A is supplied in a sterile solution for injection with a 5-mL vial nominal concentration of 20 ng/ml in 0.03 M sodium phosphate, 0.12 M sodium chloride, and 1 mg/mL HSA, at pH 6.0.

Although the composition may only contain a single type of neurotoxin, for example botulinum type A, disclosed compositions can include two or more types of neurotoxins, which can provide enhanced therapeutic effects in treating the disorders. For example, a composition administered to a patient can include botulinum types A and E, or A and B, or the like. Administering a single composition containing two different neurotoxins can permit the effective concentration of each of the neurotoxins to be lower than if a single neurotoxin is administered to the patient while still achieving the desired therapeutic effects. This type of “combination” composition can also provide benefits of both neurotoxins, for example, quicker effect combined with longer duration.

In embodiments, 100 units per mL is an appropriate dilution. However, in embodiments, different concentrations of neurotoxin can be employed for injections into different tissues. For example, in embodiments involving injection into the pylorus, injection of a large volume of fluid can swell the tissue, and therefore an increased neurotoxin concentration can be employed for injections into the pyloric region to minimize the total volume of the injection. Conversely, in embodiments involving injection into the fundus, a more dilute neurotoxin formulation can be used. For example, in an embodiment, injections can comprise dilution to, for example, 2.5 Units/0.1 mL and injection of:
a. 0.1 mL (2.5 Units) 2-30 times within the stomach body and fundus (total dose of 5-125 Units); or
b. 0.2 mL (5 Units), 2-20 times with the stomach body and fundus (total dose of 10-100 Units)
c. 0.5 mL (12.5 Units), 2-10 times with the stomach body and fundus (total dose of 25-125 Units)

In further embodiments, injections can comprise dilution to 5 Units/0.1 mL and injection of:
a. 0.1 mL (5 Units), 2-30 times within the stomach body and fundus for a total dose of 10-150 Units) or
b. 0.2 mL (10 Units), 2-20 times within the stomach body and fundus for a total dose of 20-200 Units)
c. 0.5 mL (25 Units), 2-10 times with the stomach body and fundus (total dose of 50-250 Units)

In embodiments, the neurotoxin, for example a botulinum type A, can be diluted to, for example, between 1 and 6 mL per 100 U, or between 1 and 5 mL per 100 U, or between 1 and 4 mL per 100 U, or between 1 and 2 mL per 100 U, or the like. In embodiments producing sensory effects, it can be preferred to use a greater dilution as compared to embodiments producing muscular effects.

The composition administered to the patient can also contain other pharmaceutically active ingredients, such as, protein receptor or ion channel modulators, in combination with the neurotoxin or neurotoxins. These modulators may contribute to the reduction in neurotransmission between the various neurons. For example, a composition may contain gamma aminobutyric acid (GABA) type A receptor modulators that enhance the inhibitory effects mediated by the GABAA receptor. The GABAA receptor inhibits neuronal activity by effectively shunting current flow across the cell membrane. GABAA receptor modulators may enhance the inhibitory effects of the GABAA receptor and reduce electrical or chemical signal transmission from the neurons. Examples of GABAA receptor modulators include benzodiazepines, such as diazepam, oxaxepam, lorazepam, prazepam, alprazolam, halazeapam, chordiazepoxide, and chlorazepate. Compositions may also contain glutamate receptor modulators that decrease the excitatory effects mediated by glutamate receptors. Examples of glutamate receptor modulators include agents that inhibit current flux through AMPA, NMDA, and/or kainate types of glutamate receptors. Further disclosed compositions comprise esketamine.

Disclosed neurotoxin compositions can be injected into the patient using a needle or a needleless device. In certain embodiments, the method comprises sub-dermally injecting the composition in the individual. For example, administering may comprise injecting the composition through a needle of, in embodiments, no greater than about 30 gauge. In embodiments, the injection should be made in a perpendicular manner using a 23 to 27 gauge sclerotherapy or similar needle with a tip length of, for example, 2-5 mm. In certain embodiments, the method comprises administering a composition comprising a botulinum toxin, for example botulinum toxin type A.

Administration of the disclosed compositions can be carried out by, for example, syringes, catheters, needles and other means for injecting. The injection can be performed on any area of the mammal's body that is in need of treatment, however disclosed embodiments contemplate injection into the patient's stomach and the vicinity thereof. The injection can be into any specific area such as the mucosa (epithelium, lamina propria, and muscular mucosae), the submucosa, or the muscularis propria (inner circular muscle layer, intermuscular space, and outer longitudinal muscle layer). Endoscopic injections are disclosed herein. Disclosed injection techniques can comprise, for example, intramuscular injections, submucosal injections, and combinations thereof. Disclosed methods can comprise intra-sphincteric injections, for example of the pyloric sphincter.

In embodiments, more than one injection and/or sites of injection may be necessary to achieve the desired result. Also, some injections, depending on the location to be injected, may require the use of fine, hollow, TEFLON®-coated needles. In embodiments, cross-linked silicone coated needles can be used, or urethane-coated needles, or the like. In certain embodiments, guided injection is employed, for example by electromyography (EMG) such as smooth muscle electromyography (SMEMG), or ultrasound, or fluoroscopic guidance or the like.

The frequency and the amount of injection under the disclosed methods can be determined based on the nature and location of the particular area and condition being treated. In certain cases, however, repeated injection may be desired to achieve optimal results. The frequency and the amount of the injection for each particular case can be determined by the person of ordinary skill in the art.

Although examples of routes of administration and dosages are provided, the appropriate route of administration and dosage are generally determined on a case by case basis by physicians in accordance with procedures specified in the neurotoxin label. For example, the route and dosage for administration of a Clostridial neurotoxin according to the present disclosed invention can be selected based upon criteria such as the solubility characteristics of the neurotoxin chosen as well as the intensity and scope of the condition being treated.

### Methods of Use

Disclosed embodiments comprise methods of treating stomach and digestive disorders, including for example disease-related gastroparesis such as diabetic gastroparesis, idiopathic gastroparesis, post-operative gastroparesis, post-viral gastroparesis, organic dyspepsia, functional dyspepsia, pyloric spasm, combinations thereof, and the like.

Disclosed embodiments comprise methods of reducing the occurrence, recurrence, duration, or severity of one or more symptoms of a stomach or digestive disorder including abdominal pain or discomfort, episodic stomach swelling, stomach pain, early fullness, abdominal bloating, early satiety, post-prandial fullness, post-prandial satiety, nausea, vomiting, acid reflux, diarrhea, problems swallowing, and combinations thereof.

Disclosed methods combine sensory-blocking and physical effects to achieve desired treatment goals. For example, sensory-blocking effects are achieved in embodiments by decreasing or modulating nerve activity, for example vagal nerve activity, through the use of specifically targeted neurotoxin administration. In embodiments, nausea is reduced by down-regulating vagal nerve feedback to brainstem centers.

Physical effects are achieved by decreasing or modulating muscular activity, for example activity of the muscles of and proximal to the stomach, through the use of specifically targeted neurotoxin administration. For example, in embodiments, pyloric activity is reduced. In embodiments, fundal distension potential is increased.

Disclosed embodiments comprise methods of diagnosis of a digestive disorder. For example, modes of diagnosis employed to screen for gastroparesis can comprise, for example, upper endoscopy, ultrasound, or barium x-rays. An upper endoscopy involves the practitioner passing a long, thin tube called an endoscope through the mouth and gently guiding it down the throat, also called the esophagus, into the stomach.

Ultrasound used for diagnosis of gastroparesis can comprise the use of sound waves to outline and define the shape of the gallbladder and pancreas.

A barium x-ray comprises fasting for 12 hours and ingestion of a barium liquid which coats the stomach, making stomach physiology visible, including any food present (which is typically not the case 12 hours after eating).

Further embodiments can comprise confirming a diagnosis of gastroparesis, for example through the use of gastric emptying scintigraphy, a breath test, or use of a “smart” pill. In gastric emptying scintigraphy, a meal that contains a small amount of a radioactive substance, called radioisotope, is ingested. The scan measures the rate of gastric emptying at 1, 2, 3, and 4 hours. When more than 10 percent of the meal is still in the stomach at 4 hours, the diagnosis of gastroparesis is considered.

In embodiments comprising a breath test, after ingestion of a meal containing a small amount of an isotope, breath samples are taken to measure the presence of the isotope in carbon dioxide, which is expelled when a person exhales. The results reveal how fast the stomach is emptying.

In embodiments comprising use of a smart pill, a small device in capsule form is swallowed. The device then moves through the digestive tract and collects information about its progress that is sent to a cell phone-sized receiver worn around the waist or neck. When the capsule is passed from the body with the stool in a couple of days, the receiver is returned to the doctor, who then analyzes the collected information.

Further disclosed embodiments comprise the identification of biomarkers associated with digestive disorders to aid in diagnosis. In embodiments, the presence of a specific biomarker, or the absence of a specific biomarker, or the ratio between two specific biomarkers can aid in assessing the condition of a patient, as well as the likelihood of a patient benefitting from treatments methods as disclosed herein.

For example, interleukin-10 and zonulin have been associated with postoperative delayed gastric emptying in critically ill surgical pediatric patients. Thus, in embodiments, identification of these biomarkers can aid in diagnosis of a digestive disorder, for example in diagnosing postoperative delayed gastric emptying.

Similarly, diabetic and idiopathic gastroparesis has been associated with loss of CD206-positive macrophages in the gastric antrum. Thus, in embodiments, loss of CD206-positive macrophages in the gastric antrum can aid in diagnosis of a digestive disorder, for example in diagnosing diabetic or idiopathic gastroparesis.

Disclose embodiments also comprise the use of medication. For example, Metoclopramide (Reglan) stimulates stomach muscle contractions to help gastric emptying. Metoclopramide also helps reduce nausea and vomiting. Erythromycin works by increasing the contractions that move food through the stomach. Domperidone works like metoclopramide to improve stomach emptying and decrease nausea and vomiting. Disclosed embodiments can comprise the use of metoclopramide, erythromycin, domperidone, or combinations thereof, in combination with at least one neurotoxin administration.

Further disclosed embodiments comprise the use of gastric electrical stimulation in combination with at least one neurotoxin administration. In embodiments comprising treatment of patients who have documented delayed gastric emptying with no evidence of abnormal obstruction, a gastric neurostimulator can be implanted to reduce or eliminate nausea and other symptoms. The device releases mild electrical pulses to help control nausea and vomiting associated with gastroparesis.

Disclosed methods comprise an injection paradigm localizing and isolating injection sites, for example circumferentially, in the pyloric region such as the pyloric sphincter, pyloric antrum, or pyloric canal, the fundus and proximal corpus on the greater curvature of the stomach, and the duodenum, administered, for example, approximately one centimeter apart, providing a therapeutically effective amount of a neurotoxin to the isolated smooth muscle that controls both the valve-like function of the pyloric sphincter allowing solid food and liquids to pass from the stomach in a controlled manner, and the controlled relaxation of the fundus in response to a meal.

Disclosed embodiments comprise methods of treating gastroparesis comprising;
a. administering a botulinum toxin dose into at least one of the pyloric sphincter, pyloric canal or the pyloric antrum (to aid in gastric emptying);
b. optionally administering a botulinum toxin dose into the duodenum (to aid in chemical digestion of and preparation for absorption in the small intestine) during the initial or follow-up treatment; and
c. optionally administering a botulinum toxin dose into the fundus (to increase accommodation potential); and
d. optionally administering a botulinum toxin dose into the proximal corpus (to increase distension potential);
e. thereby reducing the severity, occurrence, recurrence, or duration of at least one or more symptoms of the disorder including abdominal pain or discomfort, episodic stomach swelling, stomach pain, early fullness, abdominal bloating, early satiety, post-prandial fullness, post-prandial satiety, nausea, vomiting, acid reflux, diarrhea, problems swallowing, and combinations thereof.

Disclosed embodiments comprise the step of administering at least one botulinum toxin dose into the pyloric region, comprising the pyloric sphincter, pyloric canal and pyloric antrum. Embodiments comprising the step of administering at least one botulinum toxin dose into the pyloric sphincter, pyloric canal or pyloric antrum, can further comprise administration of additional botulinum doses, for example into at least one of the fundus, proximal corpus, or duodenum.

Further embodiments comprise administering at least two botulinum toxin doses into the pyloric region, comprising the pyloric sphincter, pyloric canal and pyloric antrum. Embodiments comprising the step of administering at least two botulinum toxin doses into the pyloric sphincter, pyloric canal or pyloric antrum, can further comprise administration of additional botulinum doses, for example into at least one of the following areas: stomach (fundus, proximal corpus) or duodenum.

Disclosed embodiments comprise the step of administering at least one botulinum toxin dose into the pyloric region, comprising the pyloric sphincter, pyloric canal and pyloric antrum. Further embodiments comprising the step of administering at least one botulinum toxin dose into the pyloric sphincter, pyloric canal or pyloric antrum, can comprise administration of additional botulinum doses, for example into at least two of the following area: stomach (fundus, proximal corpus) or duodenum.

Further embodiments comprise administering at least two botulinum toxin doses into the pyloric region, comprising the pyloric sphincter, pyloric canal and pyloric antrum. Further embodiments comprising the step of administering at least two botulinum toxin doses into the pyloric sphincter, pyloric canal or pyloric antrum, can comprise administration of additional botulinum doses, for example into at least two of the following areas: stomach (fundus, proximal corpus) or duodenum.

Disclosed methods further comprise methods of treating gastroparesis comprising:
a. administering a high dose of a botulinum toxin into the pyloric region;
b. administering a medium dose of a botulinum toxin into the duodenum;
c. administering a low dose of a botulinum toxin into the fundus;
d. thereby reducing the severity, occurrence, recurrence, or duration of a one or more symptoms of the disorder including abdominal pain or discomfort, episodic stomach swelling, stomach pain, early fullness, abdominal bloating, early satiety, post-prandial fullness, post-prandial satiety, nausea, vomiting, acid reflux, diarrhea, problems swallowing, and combinations thereof.

In embodiments, a high dose of a botulinum toxin is administered into the pyloric region to improve gastric emptying by reducing spasm; a medium dose of a botulinum toxin is administered into the duodenum to provide space for gastric dumping; and a low dose of a botulinum toxin is administered into the stomach fundus to allow for some gastric distension. In embodiments, a submucosal approach may be used to administer botulinum toxin to the fundus in order to limit penetration and to achieve a sensory blocking effect to reduce central nausea. In embodiments, injections into the fundus decrease vagal nerve activation to the brainstem which can reduce central nausea. In embodiments for intramuscular effects in some regions, for example, the pyloric region, the dose may be more concentrated, for example, 50 to 100 U per mL. In embodiments for intramuscular effects in some regions, for example, the duodenum or the pyloric sphincter, a dose may be less concentrated, for example 10 to 50 U per mL. In embodiments for sensory effects, for example, injections into the fundus, a more dilute dose may be used, for example, 10 to 25 U per mL.

In embodiments comprising high, medium and low botulinum toxin doses, the high dose can comprise, for example, 2-15 intramuscular injections of, for example, 10-15 units each.

In embodiments comprising high, medium and low botulinum toxin doses, the medium dose can comprise, for example, 2-15 intramuscular injections of, for example, 5-10 units each.

In embodiments comprising high, medium and low botulinum toxin doses, the low dose can comprise, for example, 2-15 submucosal injections of, for example, 4-7 units each.

Disclosed methods further comprise methods of treating gastroparesis comprising:
a. administering a high dose of a botulinum toxin into the pyloric region, wherein said high dose comprises 2-6 intramuscular injections, spaced 1 cm apart;
b. administering a medium botulinum toxin dose into the duodenum, wherein said medium dose comprises 2-8 intramuscular injections, spaced 1 cm apart; and
c. administering a low botulinum toxin dose into the fundus, wherein said low dose comprises 2-15 submucosal injections, spaced 2 cm to 4 cm apart;
d. thereby reducing the severity, occurrence, recurrence, or duration of one or more symptoms of the disorder including abdominal pain or discomfort, episodic stomach swelling, stomach pain, early fullness, abdominal bloating, early satiety, post-prandial fullness, post-prandial satiety, nausea, vomiting, acid reflux, diarrhea, problems swallowing, and combinations thereof.

Further embodiments comprise injection of the duodenum to decrease Cholecystokinin (CCK) levels to reduce pyloric spasm. CCK is a neuropeptide and gut hormone that regulates pancreatic enzyme secretion and gastrointestinal motility, and acts as a satiety signal. It is released simultaneously from intestinal cells and neurons in response to a meal, and thus provides both sensory and muscular effects.

In embodiments, disclosed methods comprise, for example, an injection paradigm localizing and isolating, for example, 2-8 injection sites circumferentially in the pyloric sphincter. Disclosed embodiments can further comprise 2-30 injection sites on the pyloric antrum, along the pyloric canal into the pyloric sphincter, and duodenum.

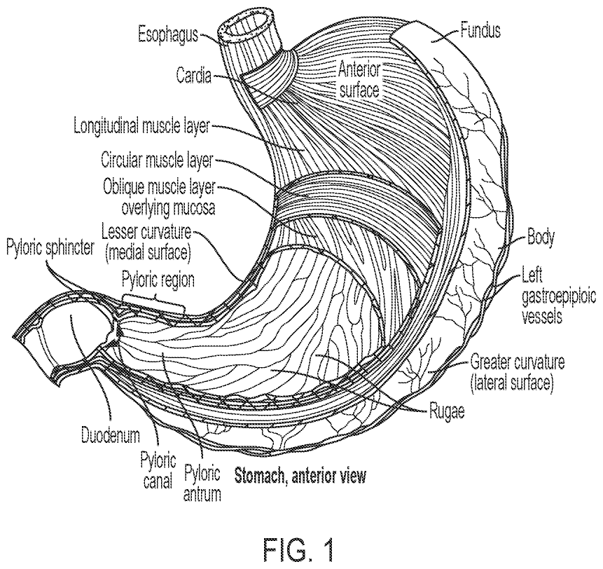

Disclosed embodiments can further comprise 2-30 injection sites on the fundus and proximal corpus on the greater curvature of the stomach (the pyloric sphincter and fundus are shown in FIG. 1) administered approximately one centimeter apart.

Disclosed methods comprise, for example, treating a patient suffering from gastroparesis with an injection paradigm localizing and isolating injection sites circumferentially, for example in the pyloric region comprising the pyloric sphincter, the pyloric canal and the pyloric antrum. Disclosed embodiments can comprise multiple injections, for example, between 1 and 5 injection sites, between 5 and 10 injection sites, between 5 and 15 injection sites, between 10 and 20 injection sites, or the like. In embodiments, the number of pyloric region injection sites can comprise, for example, between 3 and 5 injection sites, between 4 and 6 injection sites, between 5 and 7 injection sites, between 6 and 8 injection sites, between 7 and 9 injection sites, between 8 and 10 injection sites, or the like.

In embodiments, the number of pyloric region, for example the pyloric sphincter, pyloric canal and the pyloric antrum, injection sites can comprise, for example, at least 1 injection site, at least 2 injection sites, at least 3 injection sites, at least 4 injection sites, at least 5 injection sites, at least 6 injection sites, at least 7 injection sites, at least 8 injection sites, at least 9 injection sites, at least 10 injection sites, or the like.

In embodiments, the number of pyloric region, for example the pyloric sphincter, pyloric canal and the pyloric antrum, injection sites can comprise, for example, not more than 1 injection site, not more than 2 injection sites, not more than 3 injection sites, not more than 4 injection sites, not more than 5 injection sites, not more than 6 injection sites, not more than 7 injection sites, not more than 8 injection sites, not more than 9 injection sites, not more than 10 injection sites, or the like.

In embodiments, the injections to the pyloric region, for example the pyloric sphincter, pyloric canal and the pyloric antrum, are spaced, for example, 2 mm apart, 3 mm apart, 4 mm apart, 5 mm apart, 6 mm apart, 7 mm apart, 8 mm apart, 9 mm apart, 10 mm apart, 11 mm apart, 12 mm apart, 13 mm apart, 14 mm apart, 15 mm apart, 20 mm apart, 25 mm apart, 30 mm apart, or the like.

In embodiments, the injections to the pyloric region, for example the pyloric sphincter, pyloric canal and the pyloric antrum, are spaced, for example, at least 2 mm apart, at least 3 mm apart, at least 4 mm apart, at least 5 mm apart, at least 6 mm apart, at least 7 mm apart, at least 8 mm apart, at least 9 mm apart, at least 10 mm apart, at least 15 mm apart, at least 20 mm apart, at least 25 mm apart, at least 30 mm apart, or the like.

In embodiments, the injections to the pyloric region, for example the pyloric sphincter, pyloric canal and the pyloric antrum, are spaced, for example, not more than 2 mm apart, not more than 3 mm apart, not more than 4 mm apart, not more than 5 mm apart, not more than 6 mm apart, not more than 7 mm apart, not more than 8 mm apart, not more than 9 mm apart, not more than 10 mm apart, not more than 15 mm apart, not more than 20 mm apart, not more than 25 mm apart, not more than 30 mm apart, or the like.

In embodiments, the depth of each pyloric region injection should be, for example, 1 mm, 2 mm, 3 mm, 4 mm, 5 mm, 6 mm, 7 mm, 8 mm, 9 mm, 10 mm, or the like.

In embodiments, the depth of each injection to the pyloric region, for example the pyloric sphincter, pyloric canal or the pyloric antrum, injection should be, for example, between 1 and 4 mm into the smooth muscle of the treatment site, between 2 and 5 mm into the smooth muscle of the treatment site, between 3 and 6 mm into the smooth muscle of the treatment site, between 4 and 7 mm into the smooth muscle of the treatment site. In embodiments, the injection should be made in a perpendicular manner using a 23 to 27 gauge sclerotherapy or similar needle with a tip length of, for example, 2-5 mm, advanced through an endoscope in order to maximize the therapeutic effect and ensure safety. In embodiments, a “gauge” needle can be used to ensure appropriate injection depth. For example, in embodiments, the tip length of a disclosed gauge needle cans be, for example, 2-5 mm. In embodiments, the injections can be, for example, intramuscular or submucosal injections.

In embodiments, the spacing between the pyloric region injection sites can be uniform. In embodiments, the spacing between injection sites can be non-uniform, for example the sites can be adjusted to conform with the patient's specific anatomy.

Disclosed embodiments can further comprise additional injection sites on the fundus and proximal corpus on the greater curvature of the stomach. For example, disclosed embodiments comprise 1-20 injection sites on the fundus and proximal corpus, 5-25 injection sites on the fundus and proximal corpus, 10-30 injection sites on the fundus and proximal corpus, 15-35 injection sites on the fundus and proximal corpus, 20-40 injection sites on the fundus and proximal corpus, 25-45 injection sites on the fundus and proximal corpus, or the like.

Disclosed embodiments comprise, for example, at least 2 injection sites on the fundus and proximal corpus on the greater curvature of the stomach, at least 4 injection sites on the fundus and proximal corpus, at least 6 injection sites on the fundus and proximal corpus, at least 8 injection sites on the fundus and proximal corpus, at least 12 injection sites on the fundus and proximal corpus, at least 16 injection sites on the fundus and proximal corpus, at least 20 injection sites on the fundus and proximal corpus, at least 24 injection sites on the fundus and proximal corpus, at least 28 injection sites on the fundus and proximal corpus, at least 32 injection sites on the fundus and proximal corpus, at least 36 injection sites on the fundus and proximal corpus, at least 40 injection sites on the fundus and proximal corpus, or the like.

Disclosed embodiments comprise, for example, not more than 4 injection sites on the fundus and proximal corpus on the greater curvature of the stomach, not more than 6 injection sites on the fundus and proximal corpus, not more than 8 injection sites on the fundus and proximal corpus, not more than 12 injection sites on the fundus and proximal corpus, not more than 16 injection sites on the fundus and proximal corpus, not more than 20 injection sites on the fundus and proximal corpus, not more than 24 injection sites on the fundus and proximal corpus, not more than 28 injection sites on the fundus and proximal corpus, not more than 32 injection sites on the fundus and proximal corpus, not more than 36 injection sites on the fundus and proximal corpus, not more than 40 injection sites on the fundus and proximal corpus, or the like.

In embodiments, the injections on the fundus and proximal corpus are spaced, for example, 2 mm apart, 3 mm apart, 4 mm apart, 5 mm apart, 6 mm apart, 7 mm apart, 8 mm apart, 9 mm apart, 10 mm apart, 11 mm apart, 12 mm apart, 13 mm apart, 14 mm apart, 15 mm apart, 20 mm apart, 25 mm apart, 30 mm apart, or the like.

In embodiments, the injections on the fundus and proximal corpus are spaced, for example, at least 2 mm apart, at least 3 mm apart, at least 4 mm apart, at least 5 mm apart, at least 6 mm apart, at least 7 mm apart, at least 8 mm apart, at least 9 mm apart, at least 10 mm apart, at least 15 mm apart, at least 20 mm apart, at least 25 mm apart, at least 30 mm apart, or the like.

In embodiments, the injections on the fundus and proximal corpus are spaced, for example, not more than 2 mm apart, not more than 3 mm apart, not more than 4 mm apart, not more than 5 mm apart, not more than 6 mm apart, not more than 7 mm apart, not more than 8 mm apart, not more than 9 mm apart, not more than 10 mm apart, not more than 15 mm apart, not more than 20 mm apart, not more than 25 mm apart, not more than 30 mm apart, or the like.

In embodiments, the spacing between the fundus and proximal corpus injection sites can be uniform. In embodiments, the spacing between injection sites can be non-uniform, for example the sites can be adjusted to conform with the patient's specific anatomy.

In embodiments, the depth of each fundus and proximal corpus injection should be, for example, 1 mm, 2 mm, 3 mm, 4 mm, 5 mm, 6 mm, 7 mm, 8 mm, 9 mm, 10 mm, or the like. In embodiments, a “gauge” needle can be used to ensure appropriate injection depth.

In embodiments, the depth of each fundus and proximal corpus injection should be, for example, between 1 and 4 mm into the smooth muscle of the treatment site, between 2 and 5 mm into the smooth muscle of the treatment site, between 3 and 6 mm into the smooth muscle of the treatment site, between 4 and 7 mm into the smooth muscle of the treatment site. In embodiments, the injection should be made in a perpendicular manner using a 23 to 27 gauge sclerotherapy or similar needle with a tip length of, for example, 2-5 mm, advanced through an endoscope in order to maximize the therapeutic effect and ensure safety. In embodiments, injection to the fundus and proximal corpus can comprise intramuscular or submucosal injections.

Disclosed embodiments further comprise additional injection sites on the duodenum. For example, disclosed embodiments comprise 1-20 injection sites on the duodenum, 5-25 injection sites on the duodenum, or the like.

Disclosed embodiments comprise, for example, at least 2 injection sites on the duodenum, at least 4 injection sites on the duodenum, at least 6 injection sites on the duodenum, at least 8 injection sites on the duodenum, at least 12 injection sites on the duodenum, or the like.

Disclosed embodiments comprise, for example, not more than 4 injection sites on the duodenum, not more than 6 injection sites on the duodenum, not more than 8 injection sites on the duodenum, not more than 12 injection sites on the duodenum, not more than 16 injection sites on the duodenum, not more than 20 injection sites on the duodenum, not more than 24 injection sites on the duodenum, not more than 28 injection sites on the duodenum, not more than 32 injection sites on the duodenum, or the like.

In embodiments, the injections on the duodenum are spaced, for example, 2 mm apart, 3 mm apart, 4 mm apart, 5 mm apart, 6 mm apart, 7 mm apart, 8 mm apart, 9 mm apart, 10 mm apart, 11 mm apart, 12 mm apart, 13 mm apart, 14 mm apart, 15 mm apart, 20 mm apart, 25 mm apart, 30 mm apart, or the like.

In embodiments, the injections on the duodenum are spaced, for example, at least 2 mm apart, at least 3 mm apart, at least 4 mm apart, at least 5 mm apart, at least 6 mm apart, at least 7 mm apart, at least 8 mm apart, at least 9 mm apart, at least 10 mm apart, at least 15 mm apart, at least 20 mm apart, at least 25 mm apart, at least 30 mm apart, or the like.

In embodiments, the injections on the duodenum are spaced, for example, not more than 2 mm apart, not more than 3 mm apart, not more than 4 mm apart, not more than 5 mm apart, not more than 6 mm apart, not more than 7 mm apart, not more than 8 mm apart, not more than 9 mm apart, not more than 10 mm apart, not more than 15 mm apart, not more than 20 mm apart, not more than 25 mm apart, not more than 30 mm apart, or the like.

In embodiments, the spacing between the duodenum injection sites can be uniform. In embodiments, the spacing between injection sites can be non-uniform, for example the sites can be adjusted to conform with the patient's specific anatomy.

In embodiments, the depth of each duodenum injection should be, for example, 1 mm, 2 mm, 3 mm, 4 mm, 5 mm, 6 mm, 7 mm, 8 mm, 9 mm, 10 mm, or the like.

In embodiments, the depth of each duodenum injection should be, for example, between 1 and 4 mm into the smooth muscle of the treatment site, between 2 and 5 mm into the smooth muscle of the treatment site, between 3 and 6 mm into the smooth muscle of the treatment site, between 4 and 7 mm into the smooth muscle of the treatment site. In embodiments, the injection should be made in a perpendicular manner using a 23 to 27 gauge sclerotherapy or similar needle with a tip length of, for example, 2-5 mm, advanced through an endoscope in order to maximize the therapeutic effect and ensure safety.

Disclosed embodiments provide a therapeutically effective amount of a neurotoxin to the isolated smooth muscle that controls both the valve-like function of the pyloric sphincter allowing solid food and liquids to pass from the stomach in a controlled manner, and the controlled relaxation of the fundus in response to a meal, preventing early satiety and other cardinal signs and symptoms of gastroparesis.

Disclosed methods further comprise methods of treating gastroparesis comprising the steps of administering a first botulinum toxin dose into the pyloric sphincter; administering a second botulinum toxin dose in the pyloric region including the pyloric canal and/or the pyloric antrum; and administering a third botulinum toxin dose into the fundus and proximal corpus; thereby reducing the severity of at least one or more symptoms of the disorder including abdominal pain or discomfort, episodic stomach swelling, stomach pain, early fullness, abdominal bloating, early satiety, post-prandial fullness, post-prandial satiety, nausea, vomiting, acid reflux, diarrhea, problems swallowing, and combinations thereof.

Disclosed methods further comprise methods of treating a digestive disorder, for example gastroparesis, comprising administering a first botulinum toxin dose into the pyloric sphincter; administering a second botulinum toxin dose in the pyloric region including the pyloric canal and/or the pyloric antrum; and administering a third botulinum toxin dose into the duodenum; thereby reducing the severity of at least one or more symptoms of the disorder including abdominal pain or discomfort, episodic stomach swelling, stomach pain, early fullness, abdominal bloating, early satiety, post-prandial fullness, post-prandial satiety, nausea, vomiting, acid reflux, diarrhea, problems swallowing, and combinations thereof.

Disclosed embodiments can also comprise avoidance of injection into a specific anatomical site. For example, in embodiments, at least one of the fundus, proximal corpus, pyloric antrum, pyloric sphincter, pyloric canal, and duodenum is not injected. In embodiments, at least two of the fundus, proximal corpus, pyloric antrum, pyloric sphincter, pyloric canal, and duodenum are not injected. In embodiments, at least three of the fundus, proximal corpus, pyloric antrum, pyloric sphincter, pyloric canal, and duodenum are not injected.

In embodiments, disclosed methods can comprise an injection paradigm as described in Table 1:

| | | Total | | | | |
|---|---|---|---|---|---|---|
| | Total Dose | Volume/ | Number of | Dose/Site | Concentration/ | Route of |
| Region | ABP-450 | Vol/inj | injections/Site | Volume/inj. | injection | injection |
| Pyloric | Placebo | 4 ml | 4 | 1 ml/injection | | IM |
| Sphincter | | 1 ml/inj | | | | |
| Pyloric | 50 U | 4 ml | 4 | 12.5 U/injection | 12.5 U/ml | IM |
| Sphincter | | 1 ml/inj | | 1 ml/injection | | |
| Pyloric | 100 U | 4 ml | 4 | 25 U/injection | 25 U/ml | IM |
| Sphincter | | 1 ml/inj | | 1 ml/injection | | |
| Pyloric | 200 U | 4 ml | 4 | 50 U/injection | 50 U/ml | IM |
| Sphincter | | 1 ml/inj | | 1 ml/injection | | |

For example, disclosed embodiments can comprise intramuscular injections into the pyloric sphincter. In embodiments, the pyloric sphincter can be injected at between 1 and 6 sites, or 2 and 6, or 3 and 5 sites, or the like. In embodiments, the pyloric sphincter can be injected, for example, at 2, 3, 4, 5, or 6 sites. In embodiments, the pyloric sphincter can be injected at 2 sites, 1 injection in each half of the pyloric sphincter. In embodiments, the pyloric sphincter can be injected at 4 sites, 1 injection in each quadrant of the pyloric sphincter. In embodiments, the pyloric sphincter can be injected at 6 sites, 3 injections spaced equally apart in each half of the pyloric sphincter. In embodiments, the pyloric sphincter injections can comprise a total volume of between 1 and 6 mL, or 3 and 5 mL. In embodiments, the pyloric sphincter injections can comprise a total volume of 1 mL, 2 mL, 3 mL, 4 mL, 5 mL, 6 mL, or the like. In embodiments, the pyloric sphincter injections can comprise a neurotoxin dose, such as a botulinum type A, per injection of, for example, 10 Units, 12.5 Units, 15 Units, 17.5 Units, 20 Units, 25 Units, 30 Units, 35 Units, 40 Units, 45 Units, 50 Units, 55 Units, 60 Units, 65 Units, 75 Units, 80 Units, 85 Units, 90 Units, 95 Units, 100 Units or the like.

In embodiments, the pyloric sphincter injections can comprise a total neurotoxin dose, such as a botulinum type A, of between, for example, 25 and 300 Units, or 50 and 200 Units, or the like. In embodiments, the pyloric sphincter injections can comprise a total dose of, for example, 25 Units, 50 Units, 100 Units, 150 Units, 200 Units, 250 Units, 300 Units or the like. In embodiments, the dilution of the administered neurotoxin, such as a botulinum type A, for example, can be 10 Units per mL, 12.5 Units per mL, 15 Units per mL, 17.5 Units per mL, 20 Units per mL, 25 Units per mL, 30 Units per mL, 35 Units per mL, 40 Units per mL, 45 Units per mL, 50 Units per mL, 55 Units per mL, 60 Units per mL, 65 Units per mL, or the like.

Further disclosed embodiments can comprise the injection paradigm of Table 2:

| | | [symbol] | | | | [symbol] |
|---|---|---|---|---|---|---|
| [symbol] | [symbol] | [symbol] | [symbol] | [symbol] | [symbol] | [symbol] |
| [symbol] | [symbol] | [symbol] | [symbol] | | [symbol] | [symbol] |
| [symbol] | [symbol] | [symbol] | [symbol] | [symbol] | [symbol] | [symbol] |
| [symbol] | | | | [symbol] | | |
| [symbol] | [symbol] | [symbol] | [symbol] | [symbol] | [symbol] | [symbol] |
| [symbol] | | [symbol] | [symbol] | | [symbol] | [symbol] |
| | | [symbol] | [symbol] | | [symbol] | [symbol] |
| | | [symbol] | | | | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| | [symbol] | [symbol] | [symbol] | [symbol] | | [symbol] |
| | | [symbol] | [symbol] | | | |
| | | | [symbol] | | | |
| | | | [symbol] | | | |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| | [symbol] | [symbol] | [symbol] | [symbol] | [symbol] | [symbol] |
| | | [symbol] | [symbol] | | | |
| | | | [symbol] | | | |
| | | | [symbol] | | | |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| | [symbol] | [symbol] | [symbol] | [symbol] | [symbol] | [symbol] |
| | | [symbol] | [symbol] | | | |
| | | | [symbol] | | | |
| | | | [symbol] | | | |

[symbol]

indicates data missing or illegible when filed

For example, disclosed methods can comprise intramuscular injection into the pyloric sphincter of, for example, between 25 and 350 Units, or between 250 to 350 Units. For example, in an embodiment, the pyloric sphincter can be injected with 50 Units, 100 Units, 200 Units, 250 Units, 300 Units, or 350 Units. In embodiments, the injection volume per site into the pyloric sphincter can comprise 1 mL, 2 mL, 3 mL, 4 mL, 5 mL, or the like. In embodiments, the total injection volume into the pyloric sphincter can comprise 2 mL, 3 mL, 4 mL, 5 mL, or the like. In embodiments, the number of injection sites into the pyloric sphincter can between, for example, 1 and 6, 2 and 6, or 3 and 5, or the like. In embodiments, the number of Units per injection can be, for example, between 50 and 100 Units. For example, in an embodiment, the number of Units per injection can be 50 Units, 75 Units, 100 Units, or the like.

Further disclosed embodiments comprise intramuscular injection into the pyloric sphincter combined with intramuscular or submucosal injection to the fundus and intramuscular or submucosal injection to the duodenum. For example, in methods comprising injection into the pyloric sphincter, fundus, and duodenum, injection into the pyloric sphincter can comprise, for example, intramuscular injection into the pyloric sphincter of, for example, between 50 and 250 Units. For example, in an embodiment, the pyloric sphincter can be injected with 50 Units, 100 Units, 150 Units, 200 Units, or 250 Units. In an embodiment comprising injection into the pyloric sphincter combined with intramuscular or submucosal injection to the fundus and intramuscular or submucosal injection to the duodenum, the dosage administered to the pyloric sphincter can be 50 Units, 100 Units or 200 Units.

In embodiments comprising injection into the pyloric sphincter combined with intramuscular or submucosal injection to the fundus and intramuscular or submucosal injection to the duodenum, the total injection volume into the pyloric sphincter can comprise 2 mL, 3 mL, 4 mL, 5 mL, 6 mL, or the like. For example, the total volume can be 4 mL. In embodiments comprising injection into the pyloric sphincter combined with intramuscular or submucosal injection to the fundus and intramuscular or submucosal injection to the duodenum, the volume per injection into the pyloric sphincter can comprise 1 mL, 2 mL, 3 mL or the like. For example, the volume per injection can be 1 mL.

In embodiments comprising injection into the pyloric sphincter combined with intramuscular or submucosal injection to the fundus and intramuscular or submucosal injection to the duodenum, the number of injection sites into the pyloric sphincter can between, for example, 1 and 6, or 2 and 6, or 3 and 5, or the like. In embodiments, the number of injections into the pyloric sphincter can be 4 injections, for example 1 injection into each quadrant of the pyloric sphincter.

In embodiments, the number of Units per injection into the pyloric sphincter can be, for example, between 10 and 75 Units. For example, in an embodiment, the number of Units per injection can be 10 Units, 12.5 Units, 25 Units, 50 Units, 75 Units, or the like. In embodiments, the concentration of the neurotoxin injected into the pyloric sphincter can be between 10 and 100 Units per mL, for example 12.5 Units per mL, 25 Units per mL, or 50 Units per mL.

In further disclosed embodiments comprising intramuscular injection into the pyloric sphincter combined with intramuscular or submucosal injection to the fundus and intramuscular or submucosal injection to the duodenum, the injections to the fundus can comprise a total dose of, for example, between 25 and 75 Units, for example 50 Units. In embodiments, the total injection volume into the fundus can comprise 2 mL, 3 mL, 4 mL, 5 mL, 6 mL, or the like. In embodiments comprising injection into the pyloric sphincter combined with intramuscular or submucosal injection to the fundus and intramuscular or submucosal injection to the duodenum, the volume per injection into the fundus can comprise 1 mL, 2 mL, 3 mL or the like. For example, the volume per injection can be 1 mL.

In embodiments comprising injection into the pyloric sphincter combined with intramuscular or submucosal injection to the fundus and intramuscular or submucosal injection to the duodenum, the number of injection sites into the fundus can be between, for example, 2 and 6, or 3 and 5, or the like. In embodiments, the number of injections into the fundus can be 4 injections, for example 4 injections spaced 2 cm apart.

In embodiments, the number of Units per injection into the fundus can be, for example, between 5 and 20 Units. For example, in an embodiment, the number of Units per injection can be 5 Units, 12.5 Units, 20 Units, or the like. In embodiments, the concentration of the neurotoxin injected into the fundus can be between 5 and 50 Units per mL, for example 12.5 Units per mL.

In further disclosed embodiments comprising intramuscular injection into the pyloric sphincter combined with intramuscular or submucosal injection to the fundus and intramuscular or submucosal injection to the duodenum, the injections to the duodenum can comprise a total dose of, for example, between 25 and 75 Units, for example 50 Units. In embodiments, the total injection volume into the duodenum can comprise 2 mL, 3 mL, 4 mL, 5 mL, 6 mL, or the like. In embodiments comprising injection into the pyloric sphincter combined with intramuscular or submucosal injection to the fundus and intramuscular or submucosal injection to the duodenum, the volume per injection into the duodenum can comprise 1 mL, 2 mL, 3 mL or the like. For example, the volume per injection can be 1 mL. In embodiments, the concentration of the neurotoxin injected into the duodenum can be between 5 and 50 Units per mL, for example 12.5 Units per mL.

In embodiments comprising intramuscular injection into the pyloric sphincter combined with intramuscular or submucosal injection to the fundus and intramuscular or submucosal injection to the duodenum, the number of injection sites into the duodenum can be between, for example, 2 and 6, or 3 and 5, or the like. In embodiments, the number of injections into the duodenum can be 4 injections, for example 4 injections into the proximal duodenum spaced up to 10 cm from the pyloric sphincter.

In embodiments, the number of Units per injection into the duodenum can be, for example, between 5 and 50 Units. For example, in an embodiment, the number of Units per injection can be 5 Units, 12.5 Units, 20 Units, 25 Units, 30 Units, 35 Units, 40 Units, 45 Units, 50 Units, or the like. In embodiments, the concentration of the neurotoxin injected into the duodenum can be between 5 and 50 Units per mL, for example 12.5 Units per mL.

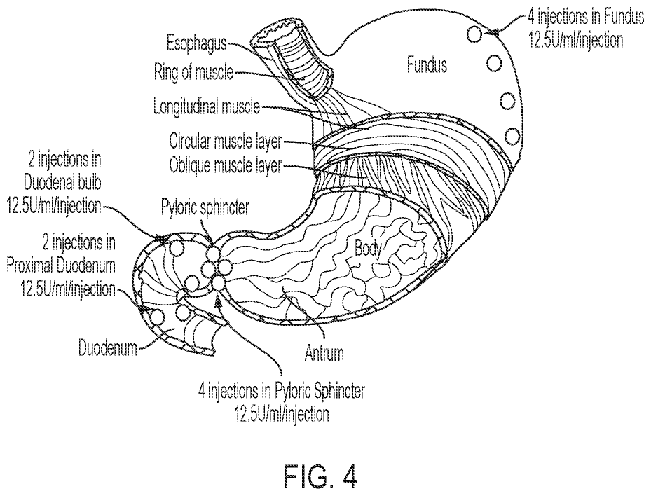

Further disclosed embodiments comprise intramuscular injection into the pyloric sphincter combined with intramuscular or submucosal injection to the fundus and intramuscular or submucosal injection to the duodenum (as shown in FIG. 4). For example, in methods comprising injection into the pyloric sphincter, fundus, and duodenum, injection into the pyloric sphincter can comprise, for example, intramuscular injection into the pyloric sphincter of, for example, between 25 and 300 Units. For example, in an embodiment, the pyloric sphincter can be injected with 25 Units, 50 Units, 75 Units, 100 Units, 150 Units, 200 Units, 250 Units, or 300 Units. In an embodiment comprising injection into the pyloric sphincter combined with intramuscular or submucosal injection to the fundus and intramuscular or submucosal injection to the duodenum, the dosage administered to the pyloric sphincter can be 50 Units, 100 Units, 150 Units or 200 Units.

In embodiments comprising injection into the pyloric sphincter combined with intramuscular or submucosal injection to the fundus and intramuscular or submucosal injection to the duodenum, the total injection volume into the pyloric sphincter can comprise 2 mL, 3 mL, 4 mL, 5 mL, 6 mL, or the like. For example, the total injection volume can be 4 mL. In embodiments comprising injection into the pyloric sphincter combined with intramuscular or submucosal injection to the fundus and intramuscular or submucosal injection to the duodenum, the volume per injection into the pyloric sphincter can comprise 1 mL, 2 mL, 3 mL or the like. For example, the volume per injection can be 1 mL.

In embodiments comprising injection into the pyloric sphincter combined with intramuscular or submucosal injection to the fundus and intramuscular or submucosal injection to the duodenum, the number of injection sites into the pyloric sphincter can between, for example, 2 and 6, or 3 and 5, or the like. In embodiments, the number of injections into the pyloric sphincter can be 4 injections, for example 1 injection into each quadrant of the pyloric sphincter.

In embodiments, the number of Units per injection into the pyloric sphincter can be, for example, between 5 and 75 Units. For example, in an embodiment, the number of Units per injection can be 5 Units, 10 Units, 12.5 Units, 15 Units, 20 Units, 25 Units, 50 Units, 75 Units or the like. For example, the number of Units per injection can be 12.5 Units, 25 Units, 50 Units or 75 Units. In embodiments, the concentration of the neurotoxin injected into the pyloric sphincter can be between 5 and 100 Units per mL, for example between 10 and 75 Units per mL, or 12.5 Units per mL, 25 Units per mL, 50 Units per mL, or 75 Units per mL.

In further disclosed embodiments comprising intramuscular injection into the pyloric sphincter combined with intramuscular or submucosal injection to the fundus and intramuscular or submucosal injection to the duodenum, the injections to the fundus can comprise a total dose of, for example, between 25 and 75 Units, for example 50 Units. In embodiments, the total injection volume into the fundus can comprise 2 mL, 3 mL, 4 mL, 5 mL, 6 mL, or the like. In embodiments comprising injection into the pyloric sphincter combined with intramuscular or submucosal injection to the fundus and intramuscular or submucosal injection to the duodenum, the volume per injection into the fundus can comprise 1 mL, 2 mL, 3 mL or the like. For example, the volume per injection can be 1 mL.

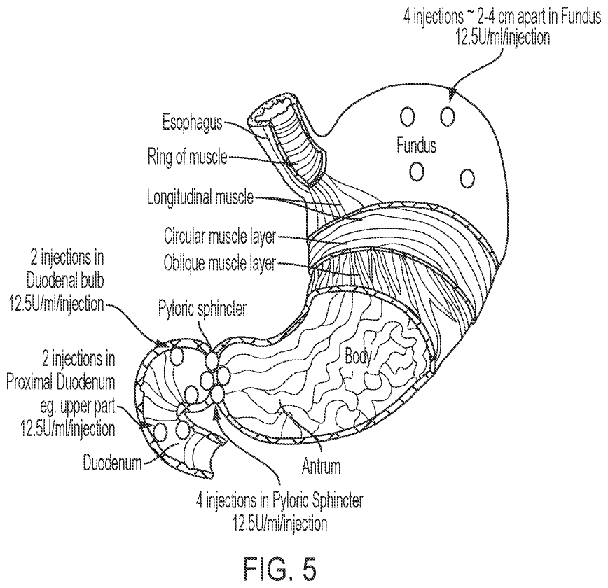

In embodiments comprising injection into the pyloric sphincter combined with intramuscular or submucosal injection to the fundus and intramuscular or submucosal injection to the duodenum, the number of injection sites into the fundus can be between, for example, 2 and 6, or 3 and 5, or the like. In embodiments, the number of injections into the fundus can be 4 injections, for example 4 injections spaced 1 cm to 4 cm apart. For example, the 4 injections can be spaced 2 cm apart or 2 cm to 4 cm apart. For example, the injections into the fundus can be in a longitudinal pattern as shown in FIG. 4 or in a transverse pattern as shown in FIG. 5. The injections into the fundus can also be in a circumferential pattern.

In embodiments, the number of Units per injection into the fundus can be, for example, between 5 and 20 Units. For example, in an embodiment, the number of Units per injection can be 5 Units, 12.5 Units, 20 Units, or the like. In embodiments, the concentration of the neurotoxin injected into the fundus can be between 5 and 25 Units per mL, for example 12.5 Units per mL.

In further disclosed embodiments comprising intramuscular injection into the pyloric sphincter combined with intramuscular or submucosal injection to the fundus and intramuscular or submucosal injection to the duodenum, the injections to the duodenum can comprise a total dose of, for example, between 25 and 75 Units, for example 50 Units. In embodiments, the total injection volume into the duodenum can comprise 2 mL, 3 mL, 4 mL, 5 mL, 6 mL, or the like. In embodiments comprising injection into the pyloric sphincter combined with intramuscular or submucosal injection to the fundus and intramuscular or submucosal injection to the duodenum, the volume per injection into the duodenum can comprise 1 mL, 2 mL, 3 mL or the like. For example, the volume per injection into the duodenum can be 1 mL.

In embodiments comprising injection into the pyloric sphincter combined with intramuscular or submucosal injection to the fundus and intramuscular or submucosal injection to the duodenum, the number of injection sites into the duodenum can be between, for example, 2 and 6, or 3 and 5, or the like. In embodiments, the number of injections into the duodenum can be 4 injections, for example 4 injections into the proximal duodenum spaced up to 10 cm from the pyloric sphincter.

In embodiments, the number of Units per injection into the duodenum can be, for example, between 5 and 20 Units. For example, in an embodiment, the number of Units per injection can be 5 Units, 12.5 Units, 20 Units, or the like. In embodiments, the concentration of the neurotoxin injected into the duodenum can be between 5 and 50 Units per mL, for example 12.5 Units per mL.

Further disclosed embodiments comprise intramuscular injection into the pyloric sphincter combined with intramuscular or submucosal injection to the fundus and intramuscular or submucosal injection to the duodenum (as shown in FIG. 5). For example, in methods comprising injection into the pyloric sphincter, fundus, and duodenum, injection into the pyloric sphincter can comprise, for example, intramuscular injection into the pyloric sphincter of, for example, between 50 and 250 Units. For example, in an embodiment, the pyloric sphincter can be injected with 50 Units, 75 Units, 100 Units, 125 Units, 150 Units, 200 Units, or 250 Units. In an embodiment comprising injection into the pyloric sphincter combined with intramuscular or submucosal injection to the fundus and intramuscular or submucosal injection to the duodenum, the dosage administered to the pyloric sphincter can be 50 Units, 100 Units, 150 Units or 200 Units.

In embodiments comprising injection into the pyloric sphincter combined with intramuscular or submucosal injection to the fundus and intramuscular or submucosal injection to the duodenum, the total injection volume into the pyloric sphincter can comprise 2 mL, 3 mL, 4 mL, 5 mL, 6 mL, or the like. For example, the total injection volume can be 4 mL. In embodiments comprising injection into the pyloric sphincter combined with intramuscular or submucosal injection to the fundus and intramuscular or submucosal injection to the duodenum, the volume per injection into the pyloric sphincter can comprise 1 mL, 2 mL, 3 mL or the like. For example, the volume per injection can be 1 mL.

In embodiments comprising injection into the pyloric sphincter combined with intramuscular or submucosal injection to the fundus and intramuscular or submucosal injection to the duodenum, the number of injection sites into the pyloric sphincter can between, for example, 2 and 6, or 3 and 5, or the like. In embodiments, the number of injections into the pyloric sphincter can be 4 injections, for example 1 injection into each quadrant of the pyloric sphincter.

In embodiments, the number of Units per injection into the pyloric sphincter can be, for example, between 10 and 75 Units. For example, in an embodiment, the number of Units per injection can be 10 Units, 12.5 Units, 15 Units, 20 Units, 25 Units, 30 Units, 35 Units, 40 Units, 45 Units, 50 Units, 55 Units, 60 Units, 65 Units, 70 Units, 75 Units, or the like. In embodiments, the concentration of the neurotoxin injected into the pyloric sphincter can be between 10 and 100 Units per mL, for example 12.5 Units per mL, 25 Units per mL, 50 Units per mL, or 75 Units per mL.

In further disclosed embodiments comprising intramuscular injection into the pyloric sphincter combined with intramuscular or submucosal injection to the fundus and intramuscular or submucosal injection to the duodenum, the injections to the fundus can comprise a total dose of, for example, between 25 and 75 Units, for example 50 Units. In embodiments, the total injection volume into the fundus can comprise 2 mL, 3 mL, 4 mL, 5 mL, 6 mL, or the like. For example, the total injection volume can be 4 mL. In embodiments comprising injection into the pyloric sphincter combined with intramuscular or submucosal injection to the fundus and intramuscular or submucosal injection to the duodenum, the volume per injection into the fundus can comprise 1 mL, 2 mL, 3 mL or the like. For example, the volume per injection can be 1 mL.

In embodiments comprising injection into the pyloric sphincter combined with intramuscular or submucosal injection to the fundus and intramuscular or submucosal injection to the duodenum, the number of injection sites into the fundus can be between, for example, 2 and 6, or 3 and 5, or the like. In embodiments, the number of injections into the fundus can be 4 injections, for example 4 injections spaced 2 cm to 4 cm apart as shown in FIG. 5.

In embodiments, the number of Units per injection into the fundus can be, for example, between 5 and 20 Units. For example, in an embodiment, the number of Units per injection can be 5 Units, 12.5 Units, 20 Units, or the like. In embodiments, the concentration of the neurotoxin injected into the fundus can be between 5 and 25 Units per mL, for example 12.5 Units per mL.

In further disclosed embodiments comprising intramuscular injection into the pyloric sphincter combined with intramuscular or submucosal injection to the fundus and intramuscular or submucosal injection to the duodenum, the injections to the duodenum can comprise a total dose of, for example, between 25 and 75 Units, for example 50 Units. In embodiments, the total injection volume into the duodenum can comprise 2 mL, 3 mL, 4 mL, 5 mL, 6 mL, or the like. For example, the total injection volume into the duodenum can be 4 mL. In embodiments comprising injection into the pyloric sphincter combined with intramuscular or submucosal injection to the fundus and intramuscular or submucosal injection to the duodenum, the volume per injection into the duodenum can comprise 1 mL, 2 mL, 3 mL or the like. For example, the volume per injection into the duodenum can be 1 mL.

In embodiments comprising injection into the pyloric sphincter combined with intramuscular or submucosal injection to the fundus and intramuscular or submucosal injection to the duodenum, the number of injection sites into the duodenum can be between, for example, 2 and 6, or 3 and 5, or the like. In embodiments, the number of injections into the duodenum can be 4 injections, for example 4 injections into the proximal duodenum spaced up to 10 cm from the pyloric sphincter.

In embodiments, the number of Units per injection into the duodenum can be, for example, between 5 and 20 Units. For example, in an embodiment, the number of Units per injection can be 5 Units, 12.5 Units, 20 Units, or the like. In embodiments, the concentration of the neurotoxin injected into the duodenum can be between 5 and 50 Units per mL, for example 12.5 Units per mL.

Disclosed methods can further comprise a surgical procedure performed in combination with, prior to, or following administration of neurotoxin. For example, disclosed embodiments comprise injection of neurotoxin in combination with, prior to, or following a gastric peroral endoscopic myotomy (G-POEM) procedure. G-POEM is an endoscopic procedure performed by creating a short submucosal “tunnel” in the distal stomach, often along the greater curvature of the stomach. The pylorus is subsequently identified within the submucosal tunnel and divided with an electrocautery device with a goal to minimize any resistance to food passage that may have been related to pylorospasm.

Disclosed embodiments also comprise the use of electromyography (EMG) to assess the electrical activity of muscle tissue. For example, disclosed embodiments comprise use of a catheter to emplace EMG electrodes, for example “reference” and “ground” electrodes, on or about the treatment area, such as at least one of the fundus, proximal corpus, pyloric antrum, pyloric sphincter, pyloric canal, and duodenum. The use of EMG allows the provider to better-localize injection sites, and can also be used to gauge the effect of disclosed treatments. For example, in embodiments, needle electrodes can be used wherein an electrode/needle assembly is used to locate an optimal injection site and deliver the neurotoxin to the site. For example, upon micro-stimulation localization of the recording/stimulating needle electrode to the treatment area, a neurotoxin can be injected.

The catheter also provides access to the treatment area for injection. A suitable injection needle for use in catheter-based embodiments can comprise, for example, a 25 gauge stainless steel needle.

In embodiments comprising the use of EMG, a suitable catheter length can be, for example, 180 cm, 230 cm, or the like.

### Neurotoxin Dosages

In various described embodiments, the neurotoxin can be administered in an amount of between about 10⁻³ U/kg and about 30 U/kg. In an embodiment, the neurotoxin is administered in an amount of between about 10⁻² U/kg and about 25 U/kg. In another embodiment, the neurotoxin is administered in an amount of between about 10⁻¹ U/kg and about 15 U/kg. In another embodiment, the neurotoxin is administered in an amount of between about 1 U/kg and about 10 U/kg. In many instances, an administration of from about 1 unit to about 300 Units of a neurotoxin, such as a botulinum type A, provides effective therapeutic relief. In an embodiment, from about 50 Units to about 400 Units of a neurotoxin, such as a botulinum type A, can be used and in another embodiment, from about 100 Units to about 300 Units of a neurotoxin, such as a botulinum type A, can be locally administered to a target tissue.

In embodiments, administration can comprise a total dose per treatment session of about 100 Units of a botulinum neurotoxin, or about 110 Units, or about 120 Units, or about 130 Units, or about 140 Units, or about 150 Units, or about 160 Units, or about 170 Units, or about 180 Units, or about 190 Units, or about 200 Units, or about 210 Units, or about 220 Units, or about 230 Units, or about 240 Units, or about 250 Units, or about 260 Units, or about 270 Units, or about 280 Units, or about 290 Units, or about 300 Units, or about 320 Units, or about 340 Units, or about 360 Units, or about 380 Units, or about 400 Units, or about 450 Units, or about 500 Units, or the like.

In embodiments, administration can comprise a total dose per treatment session of not less than 100 Units of a botulinum neurotoxin, or not less than 110 Units, or not less than 120 Units, or not less than 130 Units, or not less than 140 Units, or not less than 150 Units, or not less than 160 Units, or not less than 170 Units, or not less than 180 Units, or not less than 190 Units, or not less than 200 Units, or not less than 210 Units, or not less than 220 Units, or not less than 230 Units, or not less than 240 Units, or not less than 250 Units, or not less than 260 Units, or not less than 270 Units, or not less than 280 Units, or not less than 290 Units, or not less than 300 Units, or not less than 320 Units, or not less than 340 Units, or not less than 360 Units, or not less than 380 Units, or not less than 400 Units, or not less than 450 Units, or not less than 500 Units, or the like.

In embodiments, administration can comprise a total dose per treatment session of not more than 100 Units of a botulinum neurotoxin, or not more than 110 Units, or not more than 120 Units, or not more than 130 Units, or not more than 140 Units, or not more than 150 Units, or not more than 160 Units, or not more than 170 Units, or not more than 180 Units, or not more than 190 Units, or not more than 200 Units, or not more than 210 Units, or not more than 220 Units, or not more than 230 Units, or not more than 240 Units, or not more than 250 Units, or not more than 260 Units, or not more than 270 Units, or not more than 280 Units, or not more than 290 Units, or not more than 300 Units, or not more than 320 Units, or not more than 340 Units, or not more than 360 Units, or not more than 380 Units, or not more than 400 Units, or not more than 450 Units, or not more than 500 Units, or the like.

In embodiments, the total dose administered to the target sites in the pyloric sphincter can be, for example, about 30 Units of a botulinum neurotoxin, or about 40 Units, or about 50 Units, or about 60 Units, or about 70 Units, or about 80 Units, or about 90 Units, or about 100 Units, or about 110 Units, or about 120 Units, or about 130 Units, or about 140 Units, or about 150 Units, or about 160 Units, or about 170 Units, or about 180 Units, or about 190 Units, or about 200 Units, or about 210 Units, or about 220 Units, or about 230 Units, or about 240 Units, or about 250 Units, or about 260 Units, or about 270 Units, or about 280 Units, or about 290 Units, or about 300 Units, or the like.

In embodiments, the total dose administered to the target sites in the pyloric sphincter can be, for example, at least 30 Units of a botulinum neurotoxin, at least 40 Units, at least 50 Units, at least 60 Units, at least 70 Units, at least 80 Units, at least 90 Units, at least 100 Units, at least 110 Units, at least 120 Units, at least 130 Units, at least 140 Units, at least 150 Units, at least 160 Units, at least 170 Units, at least 180 Units, at least 190 Units, at least 200 Units, at least 210 Units, at least 220 Units, at least 230 Units, at least 240 Units, at least 250 Units, at least 260 Units, at least 270 Units, at least 280 Units, at least 290 Units, at least 300 Units, or the like.

In embodiments, the total dose administered to the target sites in the pyloric sphincter can be, for example, not more than 30 Units of a botulinum neurotoxin, not more than 40 Units, not more than 50 Units, not more than 60 Units, not more than 70 Units, not more than 80 Units, not more than 90 Units, not more than 100 Units, not more than 110 Units, not more than 120 Units, not more than 130 Units, not more than 140 Units, not more than 150 Units, not more than 160 Units, not more than 170 Units, not more than 180 Units, not more than 190 Units, not more than 200 Units, not more than 210 Units, not more than 220 Units, not more than 230 Units, not more than 240 Units, not more than 250 Units, not more than 260 Units, not more than 270 Units, not more than 280 Units, not more than 290 Units, not more than 300 Units, or the like.

In embodiments, the total dose administered to the target sites in the gastric fundus and proximal corpus can be, for example, about 30 Units of a botulinum neurotoxin, or about 40 Units, or about 50 Units, or about 60 Units, or about 70 Units, or about 80 Units, or about 90 Units, or about 100 Units, or about 110 Units, or about 120 Units, or about 130 Units, or about 140 Units, or about 150 Units, or about 160 Units, or about 170 Units, or about 180 Units, or about 190 Units, or about 200 Units, or about 210 Units, or about 220 Units, or about 230 Units, or about 240 Units, or about 250 Units, or about 260 Units, or about 270 Units, or about 280 Units, or about 290 Units, or about 300 Units, or the like.

In embodiments, the total dose administered to the target sites in the gastric fundus and proximal corpus can be, for example, at least 30 Units of a botulinum neurotoxin, at least 40 Units, at least 50 Units, at least 60 Units, at least 70 Units, at least 80 Units, at least 90 Units, at least 100 Units, at least 110 Units, at least 120 Units, at least 130 Units, at least 140 Units, at least 150 Units, at least 160 Units, at least 170 Units, at least 180 Units, at least 190 Units, at least 200 Units, at least 210 Units, at least 220 Units, at least 230 Units, at least 240 Units, at least 250 Units, at least 260 Units, at least 270 Units, at least 280 Units, at least 290 Units, at least 300 Units, or the like.

In embodiments, the total dose administered to the target sites in the gastric fundus and proximal corpus can be, for example, not more than 30 Units of a botulinum neurotoxin, not more than 40 Units, not more than 50 Units, not more than 60 Units, not more than 70 Units, not more than 80 Units, not more than 90 Units, not more than 100 Units, not more than 110 Units, not more than 120 Units, not more than 130 Units, not more than 140 Units, not more than 150 Units, not more than 160 Units, not more than 170 Units, not more than 180 Units, not more than 190 Units, not more than 200 Units, not more than 210 Units, not more than 220 Units, not more than 230 Units, not more than 240 Units, not more than 250 Units, not more than 260 Units, not more than 270 Units, not more than 280 Units, not more than 290 Units, not more than 300 Units, or the like.

In embodiments, the total dose administered to the target sites in the pyloric antrum and/or pyloric canal and/or duodenum can be, for example, about 30 Units of a botulinum neurotoxin, or about 40 Units, or about 50 Units, or about 60 Units, or about 70 Units, or about 80 Units, or about 90 Units, or about 100 Units, or about 110 Units, or about 120 Units, or about 130 Units, or about 140 Units, or about 150 Units, or about 160 Units, or about 170 Units, or about 180 Units, or about 190 Units, or about 200 Units, or about 210 Units, or about 220 Units, or about 230 Units, or about 240 Units, or about 250 Units, or about 260 Units, or about 270 Units, or about 280 Units, or about 290 Units, or about 300 Units, or the like.

In embodiments, the total dose administered to the target sites in the pyloric antrum and/or pyloric canal and/or duodenum can be, for example, at least 30 Units of a botulinum neurotoxin, at least 40 Units, at least 50 Units, at least 60 Units, at least 70 Units, at least 80 Units, at least 90 Units, at least 100 Units, at least 110 Units, at least 120 Units, at least 130 Units, at least 140 Units, at least 150 Units, at least 160 Units, at least 170 Units, at least 180 Units, at least 190 Units, at least 200 Units, at least 210 Units, at least 220 Units, at least 230 Units, at least 240 Units, at least 250 Units, at least 260 Units, at least 270 Units, at least 280 Units, at least 290 Units, at least 300 Units, or the like.

In embodiments, the total dose administered to the target sites in the pyloric antrum and/or pyloric canal and/or duodenum can be, for example, not more than 30 Units of a botulinum neurotoxin, not more than 40 Units, not more than 50 Units, not more than 60 Units, not more than 70 Units, not more than 80 Units, not more than 90 Units, not more than 100 Units, not more than 110 Units, not more than 120 Units, not more than 130 Units, not more than 140 Units, not more than 150 Units, not more than 160 Units, not more than 170 Units, not more than 180 Units, not more than 190 Units, not more than 200 Units, not more than 210 Units, not more than 220 Units, not more than 230 Units, not more than 240 Units, not more than 250 Units, not more than 260 Units, not more than 270 Units, not more than 280 Units, not more than 290 Units, not more than 300 Units, or the like.

In embodiments, administration can comprise a total dose per year of not more than 800 Units of a neurotoxin, for example botulinum type A neurotoxin, or not more than 900 Units, or not more than 1000 Units, or not more than 1200 Units, or not more than 1400 Units, or the like.

In embodiments, the dose of the neurotoxin is expressed in protein amount or concentration. For example, in embodiments the neurotoxin can be administered in an amount of between about 2 ng and 20 ng. In an embodiment, the neurotoxin is administered in an amount of between about 0.3 ng and 19 ng, about 0.4 ng and 18 ng, about 0.5 ng and 17 ng, about 0.6 ng and 16 ng, about 0.7 ng and 15 ng, about 0.8 ng and 14 ng, about 0.9 ng and 13 ng, about 1.0 ng and 12 ng, about 1.5 ng and 11 ng, about 2 ng and 10 ng, about 5 ng and 7 ng, and the like, into a target tissue such as a muscle.

Ultimately, however, both the quantity of toxin administered and the frequency of its administration will be at the discretion of the physician responsible for the treatment and will be commensurate with questions of safety and the effects produced by the toxin.

Disclosed embodiments comprise treatments that can be repeated. For example, a repeat treatment can be performed when the patient begins to experience symptoms of gastroparesis. However, preferred embodiments comprise repeating the treatment prior to the return of symptoms. Therefore, disclosed embodiments comprise repeating the treatment, for example, after 12 weeks, 14 weeks, 16 weeks, 18 weeks, 20 weeks, 22 weeks, 24 weeks, or more. Repeat treatments can comprise administration sites that differ from the administration sites used in a prior treatment.

A controlled release system can be used in the embodiments described herein to deliver a neurotoxin in vivo at a predetermined rate over a specific time period. A controlled release system can be comprised of a neurotoxin incorporated into a carrier. The carrier can be a polymer or a bio-ceramic material. The controlled release system can be injected, inserted or implanted into a selected location of a patient's body and reside therein for a prolonged period during which the neurotoxin is released by the implant in a manner and at a concentration which provides a desired therapeutic efficacy.

Polymeric materials can release neurotoxins due to diffusion, chemical reaction or solvent activation, as well as upon influence by magnetic, ultrasound or temperature change factors. Diffusion can be from a reservoir or matrix. Chemical control can be due to polymer degradation or cleavage of the drug from the polymer. Solvent activation can involve swelling of the polymer or an osmotic effect.

A kit for practicing disclosed embodiments is also encompassed by the present disclosure. The kit can comprise a 30 gauge or smaller needle and a corresponding syringe. The kit can also comprise a Clostridial neurotoxin composition, such as a botulinum type A toxin composition. The neurotoxin composition may be provided in the syringe. The composition is injectable through the needle. The kits are designed in various forms based the sizes of the syringe and the needles and the volume of the injectable composition(s) contained therein, which in turn are based on the specific deficiencies the kits are designed to treat.

In embodiments, disclosed kits can comprise a catheter, for example 180 cm or 230 cm catheters. In embodiments, the catheters can be “pre-filled” with a suitable neurotoxin formulation at a suitable concentration for the intended treatment area. In embodiments, disclosed kits can comprise neurotoxin vials of increased volume as compared to standard neurotoxin packaging. For example, in an embodiment, a disclosed kit comprises a 400 Unit vial of neurotoxin.

In embodiments, disclosed kits can comprise needle electrodes, for example needle electrodes selected from the following:

| Length | | Calibre | |
|---|---|---|---|
| mm | inches | mm | Gauge |
| 25 | 1″ | 0.30 | 30 G |
| 30 | 1.2″ | 0.36 | 28 G |
| 35 | 1.4″ | 0.40 | 27 G |
| 38 | 1.5″ | 0.45 | 26 G |
| 50 | 2″ | 0.50 | 25 G |
| 75 | 3″ | 0.55 | 24 G |

### EXAMPLES

The following non-limiting Examples are provided for illustrative purposes only in order to facilitate a more complete understanding of representative embodiments. This example should not be construed to limit any of the embodiments described in the present Specification.

### Example 1

### Treatment of Gastroparesis

A gastroparesis patient is treated via endoscopic guided injection of 50 Units total of botulinum type A into 5 injection sites spaced circumferentially in the pyloric sphincter, and 100 Units total of botulinum type A into 30 injection sites on the fundus and proximal corpus (1-4 cm apart). The patient reports a decrease in vomiting and acid reflux for 8 weeks following the treatment.

### Example 2

### Treatment of Gastroparesis

A gastroparesis patient is treated via endoscopic guided injection of 80 Units total of botulinum type E into 3 injection sites circumferentially (8 mm apart) in the pyloric sphincter, and 120 Units total of botulinum type A into 20 injection sites on the fundus and proximal corpus (spaced 1-4 cm apart). The patient reports a decrease in abdominal pain for 10 weeks following the treatment.

### Example 3

### Treatment of Gastroparesis

A gastroparesis patient is treated via endoscopic injection of 100 Units total of botulinum type A into 7 injection sites circumferentially in the pyloric sphincter, and 100 Units total of botulinum type A into 30 injection sites (1-4 cm apart) on the fundus and proximal corpus. The patient reports a decrease in bloating for 16 weeks following the treatment.

### Example 4

### Treatment of Gastroparesis

A gastroparesis patient is treated via EMG-guided endoscopic injection of 200 Units total of botulinum type A into 4 injection sites circumferentially in the pyloric sphincter, 50 Units into 4 injection sites on the duodenum and another 50 Units total of botulinum type A into 8 injection sites (1-4 cm apart) on the fundus and proximal corpus. The patient reports a decrease in vomiting and bloating for 18 weeks following the treatment.

### Example 5

### Treatment of Gastroparesis

A gastroparesis patient is treated without EMG-guided endoscopic injection of 200 Units total of botulinum type A into 4 injection sites circumferentially in the pyloric sphincter, 50 Units into 6 injection sites on the duodenum and another 50 Units total of botulinum type A into 8 injection sites 1-4 cm apart) on the fundus and proximal corpus. The patient reports a decrease in vomiting and bloating for 16 weeks following the treatment.

### Example 6

### Treatment of Gastroparesis

A gastroparesis patient is treated without EMG-guided endoscopic injection of 200 Units total of botulinum type A into 4 injection sites circumferentially in the pyloric sphincter, 50 Units into 6 injection sites on the duodenum and another 50 Units total of botulinum type A into 8 injection sites spaced 1-4 cm apart on the fundus and/or proximal corpus. The patient reports a decrease in vomiting and bloating for 16 weeks following the treatment.

### Example 7

### Treatment of Gastroparesis

A gastroparesis patient is treated without EMG-guided endoscopic injection of 200 Units total of botulinum type A into 4 injection sites circumferentially in the pyloric sphincter, 50 Units into 6 injection sites on the duodenum and another 50 Units total of botulinum type A into 7 injection sites spaced 1-4 cm apart on the fundus. The patient reports a decrease in vomiting and bloating for 14 weeks following the treatment.

### Example 8

### Treatment of Gastroparesis

A gastroparesis patient is treated via endoscopic injection of 50 Units total of botulinum type A into 5 injection sites spaced circumferentially in the pyloric sphincter, 50 Units of botulinum type A into 8 injection sites on the pyloric antrum and the pyloric canal (4 injections to each), and 100 Units total of botulinum type A into 30 injection sites on the fundus and proximal corpus, spaced 1-4 cm apart. The patient reports decreases in vomiting and acid reflux for 8 weeks following the treatment.

### Example 9

### Treatment of Gastroparesis

A gastroparesis patient is treated via endoscopic injection of 80 Units total of botulinum type A into 5 injection sites spaced circumferentially in the pyloric sphincter, 30 Units of botulinum type A into 8 injection sites on the pyloric antrum, and 70 Units total of botulinum type A into 30 injection sites on the fundus and proximal corpus spaced 1-4 cm apart. The patient reports decreases in abdominal pain and nausea.

### Example 10

### Treatment of Gastroparesis

A gastroparesis patient is treated via endoscopic injection of 80 Units total of botulinum type A into 7 injection sites spaced circumferentially in the pyloric sphincter, 40 Units of botulinum type A into 8 injection sites on the pyloric canal, and 120 Units total of botulinum type A into 8 injection sites on the fundus and proximal corpus spaced 1-4 cm apart. The patient reports decreases in bloating and diarrhea for 12 weeks following the treatment.

### Example 11

### Treatment of Gastroparesis

A gastroparesis patient is treated via endoscopic injection of 50 Units total of botulinum type B into 5 injection sites spaced circumferentially in the pyloric sphincter, 50 Units of botulinum type A into 8 injection sites on the pyloric antrum and the pyloric canal (4 injections to each), and 100 Units total of botulinum type E into 30 injection sites on the fundus spaced 1-4 cm apart.

### Example 13

### Treatment of Gastroparesis

A gastroparesis patient is treated via endoscopic injection of 50 Units total of botulinum type F into 6 injection sites spaced circumferentially in the pyloric sphincter, 60 Units of botulinum type E into 9 injection sites on the pyloric canal, and 90 Units total of botulinum type B into 30 injection sites on the fundus and proximal corpus spaced 1-4 cm apart. The patient reports a decrease in acid reflux for 14 weeks following the treatment.

### Example 14

### Treatment of Gastroparesis

A gastroparesis patient is treated via EMG-guided endoscopic injection of 100 Units total of botulinum type A into 5 injection sites spaced circumferentially in the pyloric sphincter, 50 Units of botulinum type A into 8 injection sites on the pyloric antrum and the pyloric canal (4 injections to each), and 100 Units total of botulinum type A into 30 injection sites on the duodenum. The patient reports decreases in vomiting and acid reflux for 8 weeks following the treatment.

### Example 15

### Treatment of Gastroparesis

A gastroparesis patient is treated via endoscopic injection of 120 Units total of botulinum type A into 5 injection sites spaced circumferentially in the pyloric sphincter, 30 Units of botulinum type A into 8 injection sites on the pyloric antrum, and 70 Units total of botulinum type A into 10 injection sites on the duodenum. The patient reports decreases in abdominal pain and nausea.

### Example 16

### Treatment of Gastroparesis

A gastroparesis patient is treated via endoscopic injection of 160 Units total of botulinum type A into 7 injection sites spaced circumferentially in the pyloric sphincter, 40 Units of botulinum type A into 8 injection sites on the pyloric canal, and 100 Units total of botulinum type A into 15 injection sites on the duodenum. The patient reports decreases in bloating and diarrhea for 12 weeks following the treatment.

### Example 17

### Treatment of Gastroparesis

A gastroparesis patient is treated via endoscopic injection of 150 Units total of botulinum type B into 5 injection sites spaced circumferentially in the pyloric sphincter, 50 Units of botulinum type A into 8 injection sites on the pyloric antrum and the pyloric canal (4 injections to each), and 100 Units total of botulinum type E into 20 injection sites on the duodenum. The patient reports decreases in abdominal pain and bloating for 6 weeks following the treatment.

### Example 18

### Treatment of Gastroparesis

A gastroparesis patient is treated via endoscopic injection of 200 Units total of botulinum type F into 5 injection sites spaced circumferentially in the pyloric sphincter, 50 Units of botulinum type E into 8 injection sites on the pyloric canal, and 100 Units total of botulinum type A into 20 injection sites on the duodenum. The patient reports a decrease in bloating for 8 weeks following the treatment.

### Example 19

### Treatment of Gastroparesis

A gastroparesis patient is treated via endoscopic injection of 150 Units total of botulinum type F into 6 injection sites spaced circumferentially in the pyloric sphincter, 60 Units of botulinum type E into 9 injection sites on the pyloric canal, and 90 Units total of botulinum type B into 18 injection sites on the duodenum. The patient reports a decrease in acid reflux for 14 weeks following the treatment.

### Example 20

### Treatment of Gastroparesis

A gastroparesis patient is treated via EMG-guided endoscopic injection of 150 Units total of botulinum type F into 6 injection sites spaced circumferentially in the pyloric sphincter, then 60 Units of botulinum type E into 9 injection sites on the pyloric canal, then 90 Units total of botulinum type B into 30 injection sites on the duodenum.

A follow-up treatment is performed two weeks after the initial treatment. During the follow-up treatment, 70 Units of botulinum type A is administered to 10 injection sites on the fundus spaced 1-4 cm apart.

The patient reports a decrease in nausea for 14 weeks following the treatment.

### Example 21

### Treatment of Gastroparesis

A gastroparesis patient is treated via endoscopic injection of 180 Units total of botulinum type A into 8 injection sites spaced circumferentially in the pyloric sphincter, then 60 Units of botulinum type A into 6 injection sites on the pyloric canal, then 90 Units total of botulinum type A into 30 injection sites on the duodenum.

A follow-up treatment is performed two weeks after the initial treatment. During the follow-up treatment, 50 Units of botulinum type A is administered to 16 injection sites on the fundus and proximal corpus spaced 1-4 cm apart.

The patient reports a decrease in vomiting for 10 weeks following the treatment.

### Example 22

### Treatment of Gastroparesis

A gastroparesis patient is treated via endoscopic injection of 120 Units total of botulinum type B into 12 injection sites spaced circumferentially in the pyloric sphincter, then 40 Units of botulinum type A into 8 injection sites in the pyloric canal, then 60 Units total of botulinum type A into 20 injection sites on the duodenum.

A follow-up treatment is performed three weeks after the initial treatment. During the follow-up treatment, 50 Units of botulinum type B is administered to 10 injection sites on the fundus and proximal corpus spaced 1-4 cm apart.

The patient reports a decrease in stomach pain for 20 weeks following the treatment.

### Example 23

### Treatment of Gastroparesis

A gastroparesis patient is treated via endoscopic injection of 140 Units total of botulinum type E into 16 injection sites spaced circumferentially in the pyloric sphincter, then 40 Units of botulinum type E into 8 injection sites in the pyloric canal, then 60 Units total of botulinum type E into 20 injection sites on the duodenum.

A follow-up treatment is performed three weeks after the initial treatment. During the follow-up treatment, 80 Units of botulinum type E is administered to 20 injection sites on the fundus and proximal corpus spaced 1-4 cm apart.

The patient reports a decrease in episodic stomach swelling for 14 weeks following the treatment.

### Example 24

### Treatment of Gastroparesis

A gastroparesis patient is treated via endoscopic intramuscular injection of 1) a high dose of 100 Units total of botulinum type E into 16 injection sites spaced circumferentially in the pyloric sphincter, then 20 Units of botulinum type E into 8 injection sites in the pyloric canal; 2) administering using intramuscular injection a medium dose of 60 Units total of botulinum type E into 20 injection sites on the duodenum; and 3) administering a low dose of 20 Units total of botulinum type E into 5 injection sites on the fundus using submucosal injections spaced 1-4 cm apart.

The patient reports a decrease in episodic stomach swelling for 16 weeks following the treatment.

### Example 25

### Treatment of Gastroparesis

A gastroparesis patient is treated via endoscopic intramuscular injection of 1) a high dose of 140 Units total of botulinum type B into 14 injection sites spaced circumferentially in the pyloric sphincter; 2) a medium dose of 40 Units total of botulinum type B into 20 injection sites on the duodenum; and 3) a low dose of 20 Units total of botulinum type B into 5 injection sites on the fundus using submucosal injections.

A follow-up treatment is performed three weeks after the initial treatment. During the follow-up treatment, 80 Units of botulinum type B is administered to 20 injection sites on the fundus and proximal corpus spaced 1-4 cm apart.

The patient reports a decrease in nausea for 28 weeks following the treatment.

### Example 26

### Treatment of Gastroparesis

A gastroparesis patient is treated via endoscopic injection of 1) a high dose of 180 Units total of botulinum type A into 18 injection sites spaced circumferentially in the pyloric sphincter; 2) a medium dose of 30 Units total of botulinum type A into 30 injection sites on the duodenum; and 3) a low dose of 10 Units total of botulinum type A into 5 injection sites on the fundus using submucosal injections spaced 1-4 cm apart.

The patient reports a decrease in abdominal pain for 32 weeks following the treatment.

### Example 27

### Treatment of Gastroparesis

A gastroparesis patient is treated via endoscopic injection of
1) a high dose of 180 Units total of botulinum type A into 18 injection sites spaced circumferentially in the pyloric region; 2) a medium dose of 30 Units total of botulinum type A into 10 injection sites on the duodenum; and 3) a low dose of 10 Units total of botulinum type A into 5 injection sites on the fundus using submucosal injections spaced 1-4 cm apart.

The patient reports a decrease in vomiting for 22 weeks following the treatment.

### Example 28

### Treatment of Gastroparesis

A gastroparesis patient is treated via endoscopic injection of 200 Units of botulinum type A to the pyloric sphincter. The neurotoxin is administered via 1 injection of 50 Units to each of the 4 quadrants of the pyloric sphincter. The patient also receives 50 Units of botulinum type A to the fundus, with the 4 injections spaced 1-4 cm apart, and 50 Units of botulinum type A to the duodenum, with 4 injections within 10 cm of the pyloric sphincter.

The patient reports a decrease in nausea for 16 weeks following the treatment.

### Example 29

### Treatment of Gastroparesis

A gastroparesis patient is treated via endoscopic injection of 50 Units of botulinum type A to the pyloric sphincter. The neurotoxin is administered via 1 injection of 12.5 Units to each of the 4 quadrants of the pyloric sphincter. The patient also receives 50 Units of botulinum type A to the fundus, with the 4 injections spaced 1-4 cm apart, and 50 Units of botulinum type A to the duodenum, with 4 injections within 10 cm of the pyloric sphincter.

The patient reports a decrease in vomiting for 20 weeks following the treatment.

### Example 30

### Treatment of Gastroparesis

A gastroparesis patient is treated via endoscopic injection of 100 Units of botulinum type A to the pyloric sphincter. The neurotoxin is administered via 1 injection of 25 Units to each of the 4 quadrants of the pyloric sphincter. The patient also receives 50 Units of botulinum type A to the fundus, with the 4 injections spaced 1-4 cm apart, and 50 Units of botulinum type A to the duodenum, with 4 injections within 10 cm of the pyloric sphincter.

The patient reports a decrease in abdominal pain for 16 weeks following the treatment.

In closing, it is to be understood that although aspects of the present Specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular methodology, protocol, and/or reagent, etc., described herein. As such, various modifications or changes to or alternative configurations of the disclosed subject matter can be made in accordance with the teachings herein without departing from the spirit of the present Specification. Lastly, the terminology used herein is for the purpose of describing particular embodiments only, and is not intended to limit the scope of the present disclosure, which is defined solely by the claims. Accordingly, embodiments of the present disclosure are not limited to those precisely as shown and described.

Certain embodiments are described herein, comprising the best mode known to the inventor for carrying out the methods and devices described herein. Of course, variations on these described embodiments will become apparent to those of ordinary skill in the art upon reading the foregoing description. Accordingly, this disclosure comprises all modifications and equivalents of the subject matter recited in the claims appended hereto as permitted by applicable law. Moreover, any combination of the above-described embodiments in all possible variations thereof is encompassed by the disclosure unless otherwise indicated herein or otherwise clearly contradicted by context.

Groupings of alternative embodiments, elements, or steps of the present disclosure are not to be construed as limitations. Each group member may be referred to and claimed individually or in any combination with other group members disclosed herein. It is anticipated that one or more members of a group may be comprised in, or deleted from, a group for reasons of convenience and/or patentability. When any such inclusion or deletion occurs, the Specification is deemed to contain the group as modified thus fulfilling the written description of all Markush groups used in the appended claims.

Unless otherwise indicated, all numbers expressing a characteristic, item, quantity, parameter, property, term, and so forth used in the present Specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the Specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques. Notwithstanding that the numerical ranges and values setting forth the broad scope of the disclosure are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present Specification as if it were individually recited herein.

The terms “a,” “an,” “the” and similar referents used in the context of describing the disclosure (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the disclosure and does not pose a limitation on the scope otherwise claimed. No language in the present Specification should be construed as indicating any non-claimed element essential to the practice of embodiments disclosed herein.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the present disclosure so claimed are inherently or expressly described and enabled herein.

### DISCLOSED EMBODIMENTS

Embodiment 1) A method of treating a digestive disorder comprising;
a. administering a high dose of a botulinum toxin into at least one of the pyloric sphincter, pyloric canal, or pyloric antrum;
b. administering a medium dose of a botulinum toxin into the duodenum; and
c. administering a low dose of a botulinum toxin into the fundus;
thereby reducing the severity of at least one symptom of the disorder.

Embodiment 2) The method of embodiment 1, wherein said botulinum toxin comprises botulinum toxin type A.

Embodiment 3) The method of embodiment 2, wherein said botulinum toxin comprises native immunotype A.

Embodiment 4) The method of embodiment 1, wherein said botulinum toxin comprises botulinum toxin type B, C, E, or F.

Embodiment 5) The method of embodiment 4, wherein said botulinum toxin comprises botulinum toxin type B.

Embodiment 6) The method of embodiment 4, wherein said botulinum toxin comprises botulinum toxin type C.

Embodiment 7) The method of embodiment 4, wherein said botulinum toxin comprises botulinum toxin type E.

Embodiment 8) The method of embodiment 4, wherein said botulinum toxin comprises botulinum toxin type F.

Embodiment 9) The method of embodiments 2-8, wherein said botulinum toxin is administered by injection.

Embodiment 10) The method of embodiment 9, wherein said digestive disorder comprises idiopathic gastroparesis.

Embodiment 11) The method of embodiment 9, wherein said digestive disorder comprises diabetic gastroparesis.

Embodiment 12) The method of embodiment 9, wherein said treating comprises reducing a symptom of a digestive disorder, said symptom comprising at least one of swollen stomach, gassiness, chronic abdominal pain or discomfort, episodic stomach swelling, stomach pain, early fullness, abdominal bloating, early satiety, post-prandial fullness, post-prandial satiety, nausea, vomiting, acid reflux, diarrhea, problems swallowing, and combinations thereof.

Embodiment 13) The method of embodiment 12, wherein said symptom comprises abdominal pain.

Embodiment 14) The method of embodiment 12, wherein said symptom comprises nausea.

Embodiment 15) The method of embodiment 12, wherein said symptom comprises vomiting.

Embodiment 16) The method of embodiment 12, wherein said symptom comprises diarrhea.

Embodiment 17) The method of embodiment 12, wherein said symptom comprises acid reflux.

Embodiment 18) The method of embodiment 12, wherein said symptom comprises swollen stomach.

Embodiment 19) The method of embodiment 1, wherein said high dose comprises an amount of between 120 and 200 Units.

Embodiment 20) The method of embodiment 1, wherein said medium dose comprises an amount of between 75 and 120 Units.

Embodiment 21) The method of embodiment 1, wherein said low dose comprises an amount of between 20 and 75 Units.

Embodiment 22) A method of treating a digestive disorder comprising the steps of;
a. administering a first botulinum toxin into at least one of the pyloric sphincter, pyloric canal, or pyloric antrum; and
b. optionally administering a second botulinum toxin into at least one of the fundus, proximal corpus, and duodenum;
thereby reducing the severity of at least one symptom of the disorder.

Embodiment 23) The method of embodiment 22, wherein at least one of said first and second botulinum toxins comprise botulinum toxin type A.

Embodiment 24) The method of embodiment 23, wherein at least one of said first and second botulinum toxins comprise native immunotype A.

Embodiment 25) The method of embodiment 22, wherein at least one of said first and second botulinum toxins comprise botulinum toxin type B, C, E, or F.

Embodiment 26) The method of embodiment 25, wherein at least one of said first and second botulinum toxins comprise botulinum toxin type B.

Embodiment 27) The method of embodiment 25, wherein at least one of said first and second botulinum toxins comprise botulinum toxin type C.

Embodiment 28) The method of embodiment 25, wherein at least one of said first and second botulinum toxins comprise botulinum toxin type E.

Embodiment 29) The method of embodiment 25, wherein at least one of said first and second botulinum toxins comprise botulinum toxin type F.

Embodiment 30) The method of embodiment 23, wherein at least one of said first and second botulinum toxins are administered by injection.

Embodiment 31) The method of embodiment 30, wherein said treating comprises reducing a symptom of said digestive disorder, said symptom comprising at least one of swollen stomach, gassiness, chronic abdominal pain or discomfort, episodic stomach swelling, stomach pain, early fullness, abdominal bloating, early satiety, post-prandial fullness, post-prandial satiety, nausea, vomiting, acid reflux, diarrhea, problems swallowing, and combinations thereof.

Embodiment 32) The method of embodiment 31, wherein said symptom comprises abdominal pain.

Embodiment 33) The method of embodiment 31, wherein said symptom comprises nausea.

Embodiment 34) The method of embodiment 31, wherein said symptom comprises vomiting.

Embodiment 35) The method of embodiment 31, wherein said symptom comprises diarrhea.

Embodiment 36) The method of embodiment 31, wherein said symptom comprises acid reflux.

Embodiment 37) The method of embodiment 22, wherein said first botulinum toxin is administered in an amount of between 50 and 300 Units.

Embodiment 38) The method of embodiment 22, wherein said second botulinum toxin is administered in an amount of between 50 and 200 Units.

Embodiment 39) The method of embodiment 30, wherein said injection comprises an endoscopic injection.

Embodiment 40) The method of embodiment 39, wherein said digestive disorder comprises gastroparesis.

Embodiment 41) A method of treating gastroparesis comprising the steps of;
a. administering a first botulinum toxin into the pyloric region; and
b. administering a second botulinum toxin into the duodenum;
c. administering a third botulinum toxin into the fundus, thereby reducing the severity of at least one symptom of the disorder.

Embodiment 42) The method of embodiment 41, wherein at least one of said first, second, and third botulinum toxins comprise botulinum toxin type A.

Embodiment 43) The method of embodiment 42, wherein at least one of said first, second, and third botulinum toxins comprise native immunotype A.

Embodiment 44) The method of embodiment 41, wherein at least one of said first, second, and third botulinum toxins comprise botulinum toxin type B, C, E, or F.

Embodiment 45) The method of embodiment 44, wherein at least one of said first, second, and third botulinum toxins comprise botulinum toxin type B.

Embodiment 46) The method of embodiment 44, wherein at least one of said first, second, and third botulinum toxins comprise botulinum toxin type C.

Embodiment 47) The method of embodiment 44, wherein at least one of said first, second, and third botulinum toxins comprise botulinum toxin type E.

Embodiment 48) The method of embodiment 44, wherein at least one of said first, second, and third botulinum toxins comprise botulinum toxin type F.

Embodiment 49) The method of embodiment 41, wherein at least one of said first, second, and third botulinum toxins are administered by injection.

Embodiment 50) The method of embodiment 49, wherein said treating comprises reducing a symptom of gastroparesis, said symptom comprising at least one of swollen stomach, gassiness, chronic abdominal pain or discomfort, episodic stomach swelling, stomach pain, early fullness, abdominal bloating, early satiety, post-prandial fullness, post-prandial satiety, nausea, vomiting, acid reflux, diarrhea, problems swallowing, and combinations thereof.

Embodiment 51) The method of embodiment 50, wherein said symptom comprises abdominal pain.

Embodiment 52) The method of embodiment 50, wherein said symptom comprises nausea.

Embodiment 53) The method of embodiment 50, wherein said symptom comprises vomiting.

Embodiment 54) The method of embodiment 50, wherein said symptom comprises diarrhea.

Embodiment 55) The method of embodiment 50, wherein said symptom comprises acid reflux.

Embodiment 56) The method of embodiment 41, wherein said first botulinum toxin is administered in an amount of between 50 and 200 Units.

Embodiment 57) The method of embodiment 41, wherein said second botulinum toxin is administered in an amount of between 50 and 200 Units.

Embodiment 58) The method of embodiment 41, wherein said third botulinum toxin is administered in an amount of between 50 and 200 Units.

Embodiment 59) A method for comparing the efficacy and safety of two different Botulinum toxins, comprising:
a. measuring a reduction of a gastroparesis symptom of an individual resulting from administration of a first botulinum neurotoxin;
b. measuring a reduction of a gastroparesis symptom of an individual resulting from administration of a second botulinum neurotoxin; and
c. comparing the reduction in symptoms to determine a difference between the first botulinum neurotoxin and the second botulinum neurotoxin.

Embodiment 60) A method of decreasing a sensory effect associated with gastroparesis, comprising administering a low dose of a neurotoxin to the vagus nerve in the proximal corpus, wherein said low dose comprises a dose insufficient to cause muscle paralysis.

Embodiment 61) The method of embodiments 2-8 wherein said botulinum toxin is administered by EMG-guided injections

Embodiment 62) The method of embodiment 30, wherein said injection comprises an EMG-guided endoscopic injection.

Embodiment 63) The method of embodiment 30, wherein said injection does not comprise an EMG-guided endoscopic injection.

Embodiment 64) The method of embodiment 30, wherein EMG said injection comprises an EMG-guided endoscopic injection into the pyloric region and duodenum only; and is then administered into the fundus without EMG

Embodiment 65) A method of treating gastroparesis comprising:
a. administration of between 25 and 300 Units of a botulinum toxin to the pyloric sphincter;
b. administration of between 25 and 200 Units of a botulinum toxin to the fundus; and
c. administration of between 25 and 100 Units of a botulinum toxin to the duodenum.

Embodiment 66) The method of embodiment 65, wherein said administration to the pyloric sphincter comprises 1 injection to each half of the pyloric sphincter; or 3 injections equally spaced apart to each half of the pyloric sphincter; or 1 injection to each quadrant of the pyloric sphincter.

Embodiment 67) The method of embodiment 65, wherein said administration to the fundus comprises 2-10 injections in a longitudinal, transverse or circumferential pattern spaced between 1 and 4 cm apart.

Embodiment 68) The method of embodiment 65, wherein said administration to the duodenum comprises 1 injection to each side of the duodenum bulb and between 2 and 6 injections on the duodenum no further than 10 cm from the pyloric sphincter.

Embodiment 69) The method of embodiment 65, wherein said treating comprises reducing a symptom of a digestive disorder, said symptom comprising at least one of swollen stomach, gassiness, chronic abdominal pain or discomfort, episodic stomach swelling, stomach pain, early fullness, abdominal bloating, early satiety, post-prandial fullness, post-prandial satiety, nausea, vomiting, acid reflux, diarrhea, problems swallowing, and combinations thereof.

Embodiment 70) The method of embodiment 69, wherein said symptom comprises abdominal pain.

Embodiment 71) The method of embodiment 69, wherein said symptom comprises nausea.

Embodiment 72) The method of embodiment 69, wherein said symptom comprises vomiting.

Embodiment 73) The method of embodiment 69, wherein said symptom comprises diarrhea.

Embodiment 74) The method of embodiment 69, wherein said symptom comprises acid reflux.

Embodiment 75) The method of claim 69, wherein said symptom comprises swollen stomach.

Embodiment 76) The method of embodiment 65, wherein said botulinum toxin comprises botulinum toxin type A.

Embodiment 77) The method of embodiment 65, wherein said botulinum toxin comprises botulinum toxin type B.

Embodiment 78) The method of embodiment 65, wherein said botulinum toxin comprises botulinum toxin type C.

Embodiment 79) The method of embodiment 65, wherein said botulinum toxin comprises botulinum toxin type E.

Embodiment 80) The method of embodiment 65, wherein said botulinum toxin comprises botulinum toxin type F.

Embodiment 81) The method of embodiment 65, wherein said administration to the pyloric sphincter comprises 200 Units, said administration to the fundus comprises 50 Units, and said administration to the duodenum comprises 50 Units.

Embodiment 82) The method of embodiment 65, wherein said administration to the pyloric sphincter comprises 50 Units, said administration to the fundus comprises 50 Units, and said administration to the duodenum comprises 50 Units.

Embodiment 83) The method of embodiment 65, wherein said administration to the pyloric sphincter comprises 100 Units, said administration to the fundus comprises 50 Units, and said administration to the duodenum comprises 50 Units.

1) A method of treating a digestive disorder comprising;
administering a high dose of a botulinum toxin into at least one of the pyloric sphincter, pyloric canal, or pyloric antrum;
administering a medium dose of a botulinum toxin into the duodenum; and
administering a low dose of a botulinum toxin into the fundus;
thereby reducing the severity of at least one symptom of the disorder.

2) The method of claim 1, wherein said botulinum toxin comprises botulinum toxin type A.

3) The method of claim 2, wherein said botulinum toxin comprises native immunotype A.

4) The method of claim 1, wherein said botulinum toxin comprises botulinum toxin type B, C, E, or F.

5) The method of claim 4, wherein said botulinum toxin comprises botulinum toxin type B.

6) The method of claim 4, wherein said botulinum toxin comprises botulinum toxin type C.

7) The method of claim 4, wherein said botulinum toxin comprises botulinum toxin type E.

8) The method of claim 4, wherein said botulinum toxin comprises botulinum toxin type F.

9) The method of claims 2-8, wherein said botulinum toxin is administered by injection.

10) The method of claim 9, wherein said digestive disorder comprises idiopathic gastroparesis.

11) The method of claim 9, wherein said digestive disorder comprises diabetic gastroparesis.

12) The method of claim 9, wherein said treating comprises reducing a symptom of a digestive disorder, said symptom comprising at least one of swollen stomach, gassiness, chronic abdominal pain or discomfort, episodic stomach swelling, stomach pain, early fullness, abdominal bloating, early satiety, post-prandial fullness, post-prandial satiety, nausea, vomiting, acid reflux, diarrhea, problems swallowing, and combinations thereof.

13) The method of claim 12, wherein said symptom comprises abdominal pain.

14) The method of claim 12, wherein said symptom comprises nausea.

15) The method of claim 12, wherein said symptom comprises vomiting.

16) The method of claim 12, wherein said symptom comprises diarrhea.

17) The method of claim 12, wherein said symptom comprises acid reflux.

18) The method of claim 12, wherein said symptom comprises swollen stomach.

19) The method of claim 1, wherein said high dose comprises an amount of between 120 and 200 Units.

20) The method of claim 1, wherein said medium dose comprises an amount of between 75 and 120 Units.

21) The method of claim 1, wherein said low dose comprises an amount of between 20 and 75 Units.

22) A method of treating a digestive disorder comprising the steps of;
administering a first botulinum toxin into at least one of the pyloric sphincter, pyloric canal, or pyloric antrum; and
optionally administering a second botulinum toxin into at least one of the fundus, proximal corpus, and duodenum;
thereby reducing the severity of at least one symptom of the disorder.

23) The method of claim 22, wherein at least one of said first and second botulinum toxins comprise botulinum toxin type A.

24) The method of claim 23, wherein at least one of said first and second botulinum toxins comprise native immunotype A.

25) The method of claim 22, wherein at least one of said first and second botulinum toxins comprise botulinum toxin type B, C, E, or F.

26) The method of claim 25, wherein at least one of said first and second botulinum toxins comprise botulinum toxin type B.

27) The method of claim 25, wherein at least one of said first and second botulinum toxins comprise botulinum toxin type C.

28) The method of claim 25, wherein at least one of said first and second botulinum toxins comprise botulinum toxin type E.

29) The method of claim 25, wherein at least one of said first and second botulinum toxins comprise botulinum toxin type F.

30) The method of claim 23, wherein at least one of said first and second botulinum toxins are administered by injection.

31) The method of claim 30, wherein said treating comprises reducing a symptom of said digestive disorder, said symptom comprising at least one of swollen stomach, gassiness, chronic abdominal pain or discomfort, episodic stomach swelling, stomach pain, early fullness, abdominal bloating, early satiety, post-prandial fullness, post-prandial satiety, nausea, vomiting, acid reflux, diarrhea, problems swallowing, and combinations thereof.

32) The method of claim 31, wherein said symptom comprises abdominal pain.

33) The method of claim 31, wherein said symptom comprises nausea.

34) The method of claim 31, wherein said symptom comprises vomiting.

35) The method of claim 31, wherein said symptom comprises diarrhea.

36) The method of claim 31, wherein said symptom comprises acid reflux.

37) The method of claim 22, wherein said first botulinum toxin is administered in an amount of between 50 and 300 Units.

38) The method of claim 22, wherein said second botulinum toxin is administered in an amount of between 50 and 200 Units.

39) The method of claim 30, wherein said injection comprises an endoscopic injection.

40) The method of claim 39, wherein said digestive disorder comprises gastroparesis.

41) A method of treating gastroparesis comprising the steps of;
administering a first botulinum toxin into the pyloric region; and
administering a second botulinum toxin into the duodenum;
administering a third botulinum toxin into the fundus,
thereby reducing the severity of at least one symptom of the disorder.

42) The method of claim 41, wherein at least one of said first, second, and third botulinum toxins comprise botulinum toxin type A.

43) The method of claim 42, wherein at least one of said first, second, and third botulinum toxins comprise native immunotype A.

44) The method of claim 41, wherein at least one of said first, second, and third botulinum toxins comprise botulinum toxin type B, C, E, or F.

45) The method of claim 44, wherein at least one of said first, second, and third botulinum toxins comprise botulinum toxin type B.

46) The method of claim 44, wherein at least one of said first, second, and third botulinum toxins comprise botulinum toxin type C.

47) The method of claim 44, wherein at least one of said first, second, and third botulinum toxins comprise botulinum toxin type E.

48) The method of claim 44, wherein at least one of said first, second, and third botulinum toxins comprise botulinum toxin type F.

49) The method of claim 41, wherein at least one of said first, second, and third botulinum toxins are administered by injection.

50) The method of claim 49, wherein said treating comprises reducing a symptom of gastroparesis, said symptom comprising at least one of swollen stomach, gassiness, chronic abdominal pain or discomfort, episodic stomach swelling, stomach pain, early fullness, abdominal bloating, early satiety, post-prandial fullness, post-prandial satiety, nausea, vomiting, acid reflux, diarrhea, problems swallowing, and combinations thereof.

51) The method of claim 50, wherein said symptom comprises abdominal pain.

52) The method of claim 50, wherein said symptom comprises nausea.

53) The method of claim 50, wherein said symptom comprises vomiting.

54) The method of claim 50, wherein said symptom comprises diarrhea.

55) The method of claim 50, wherein said symptom comprises acid reflux.

56) The method of claim 41, wherein said first botulinum toxin is administered in an amount of between 50 and 200 Units.

57) The method of claim 41, wherein said second botulinum toxin is administered in an amount of between 50 and 200 Units.

58) The method of claim 41, wherein said third botulinum toxin is administered in an amount of between 50 and 200 Units.

59) A method for comparing the efficacy and safety of two different Botulinum toxins, comprising:
measuring a reduction of a gastroparesis symptom of an individual resulting from administration of a first botulinum neurotoxin;
measuring a reduction of a gastroparesis symptom of an individual resulting from administration of a second botulinum neurotoxin; and
comparing the reduction in symptoms to determine a difference between the first botulinum neurotoxin and the second botulinum neurotoxin.

60) A method of decreasing a sensory effect associated with gastroparesis, comprising administering a low dose of a neurotoxin to the vagus nerve in the proximal corpus, wherein said low dose comprises a dose insufficient to cause muscle paralysis.

61) The method of claims 2-8 wherein said botulinum toxin is administered by EMG-guided injections

62) The method of claim 30, wherein said injection comprises an EMG-guided endoscopic injection.

63) The method of claim 30, wherein said injection does not comprise an EMG-guided endoscopic injection.

64) The method of claim 30, wherein EMG said injection comprises an EMG-guided endoscopic injection into the pyloric region and duodenum only; and is then administered into the fundus without EMG

65) A method of treating gastroparesis comprising:
administration of between 25 and 300 Units of a botulinum toxin to the pyloric sphincter;
administration of between 25 and 200 Units of a botulinum toxin to the fundus; and
administration of between 25 and 100 Units of a botulinum toxin to the duodenum.

66) The method of claim 65, wherein said administration to the pyloric sphincter comprises 1 injection to each half of the pyloric sphincter; or 3 injections equally spaced apart to each half of the pyloric sphincter; or 1 injection to each quadrant of the pyloric sphincter.

67) The method of claim 65, wherein said administration to the fundus comprises 2-10 injections in a longitudinal, transverse or circumferential pattern spaced between 1 and 4 cm apart.

68) The method of claim 65, wherein said administration to the duodenum comprises 1 injection to each side of the duodenum bulb and between 2 and 6 injections on the duodenum no further than 10 cm from the pyloric sphincter.

69) The method of claim 65, wherein said treating comprises reducing a symptom of a digestive disorder, said symptom comprising at least one of swollen stomach, gassiness, chronic abdominal pain or discomfort, episodic stomach swelling, stomach pain, early fullness, abdominal bloating, early satiety, post-prandial fullness, post-prandial satiety, nausea, vomiting, acid reflux, diarrhea, problems swallowing, and combinations thereof.

70) The method of claim 69, wherein said symptom comprises abdominal pain.

71) The method of claim 69, wherein said symptom comprises nausea.

72) The method of claim 69, wherein said symptom comprises vomiting.

73) The method of claim 69, wherein said symptom comprises diarrhea.

74) The method of claim 69, wherein said symptom comprises acid reflux.

75) The method of claim 69, wherein said symptom comprises swollen stomach.

76) The method of claim 65, wherein said botulinum toxin comprises botulinum toxin type A.

77) The method of claim 65, wherein said botulinum toxin comprises botulinum toxin type B.

78) The method of claim 65, wherein said botulinum toxin comprises botulinum toxin type C.

79) The method of claim 65, wherein said botulinum toxin comprises botulinum toxin type E.

80) The method of claim 65, wherein said botulinum toxin comprises botulinum toxin type F.

81) The method of claim 65, wherein said administration to the pyloric sphincter comprises 200 Units, said administration to the fundus comprises 50 Units, and said administration to the duodenum comprises 50 Units.

82) The method of claim 65, wherein said administration to the pyloric sphincter comprises 50 Units, said administration to the fundus comprises 50 Units, and said administration to the duodenum comprises 50 Units.

83) The method of claim 65, wherein said administration to the pyloric sphincter comprises 100 Units, said administration to the fundus comprises 50 Units, and said administration to the duodenum comprises 50 Units.
